(19) United States
(12) Patent Application Publication
(10) Pub. No.: US 2016/0331537 A1
(43) Pub. Date: Nov. 17, 2016

(54) MODIFIABLE IMPLANTS

(71) Applicant: Elwha LLC, Bellevue, WA (US)

(72) Inventors: Roderick A. Hyde, Redmond, WA (US); Muriel Y. Ishikawa, Livermore, CA (US); Robert Langer, Newton, MA (US); Eric C. Leuthardt, St. Louis, MO (US); Stephen L. Malaska, Redmond, WA (US); Lowell L. Wood, JR., Bellevue, WA (US)

(21) Appl. No.: 14/712,727

(22) Filed: May 14, 2015

(51) Int. Cl. A61F 2/28

(52) U.S. Cl. CPC A61F 2/28; A61F 2002/485

(57) ABSTRACT

Embodiments disclosed herein are directed to implants having a modifiable structural connectivity. A modifiable implant includes a body and a release member associated including a reactive composite material associated therewith. The structural connectivity of the implant can be modified upon activation of the release member, such as for removal of the implant. Systems and methods of using the same are disclosed.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None

### RELATED APPLICATIONS

None

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

Embodiments disclosed herein are directed to modifiable implants, and methods and systems of using the same. In an embodiment, a modifiable implant is disclosed. In an embodiment, the modifiable implant includes at least one body configured to be implanted in a subject and at least one release member disposed on at least a portion of the at least one body. In an embodiment, the at least one release member includes a reactive composite material and at least one release material associated with the reactive composite material. In an embodiment, the release material can be configured to at least partially alter at least a structural connectivity of the at least one release member.

In an embodiment, a method of removing an implant is disclosed. In an embodiment, the method includes locating an implant in a subject. In an embodiment, the implant includes at least one body and at least one release member disposed on at least a portion of the at least one body. In an embodiment, the at least one release member includes a reactive composite material and at least one release material associated with the reactive composite material. In an embodiment, the at least one release material is configured to at least partially alter the at least one release member to enable the at least one body to be removed from the subject. In an embodiment, the method further includes activating the at least one release member to facilitate removal of the at least one body from the subject.

In an embodiment, a system for modifying an implant is disclosed. In an embodiment, the system includes an implant configured to be implanted in a subject. In an embodiment, the implant includes at least one body and at least one release member disposed on at least a portion of the at least one body. In an embodiment, the at least one release member includes a reactive composite material and at least one release material associated with the reactive composite material. In an embodiment, the at least one release material is configured to at least partially alter at least a structural connectivity of the at least one release member. In an embodiment, the system further includes a stimulus source configured to provide a stimulus to the at least one release member effective to cause activation thereof.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B are isometric views of an implanted modifiable implant before and after use of an associated release member, according to an embodiment.

FIGS. 2A-2D are cross-sectional views of respective portions of release members, according to embodiments.

FIGS. 3A and 3B are schematic cross-sectional views of a release member of an associated RCM before and after use, according to an embodiment.

FIG. 3C is a schematic diagram of a release member according to an embodiment.

FIGS. 4A and 4B are schematic diagrams of a release member including cross-sectional views of an associated RCM before and after use, according to an embodiment.

FIGS. 5A and 5B are schematic diagrams of a release member including cross-sectional views of an associated RCM before and after use, according to an embodiment.

FIG. 6 is a schematic cross-sectional view of a release member including of an associated RCM before use, according to an embodiment.

FIG. 7A is an isometric view of a modifiable implant, according to an embodiment.

FIGS. 7B-7D are cross-sectional views of the modifiable implant of FIG. 7A, according to embodiments.

FIGS. 8A-8C are isometric views of modifiable implants, according to embodiments.

FIGS. 9A and 9B are isometric views of a modifiable implant before and after use, according to an embodiment.

FIG. 9C is an isometric view of a modifiable implant, according to an embodiment.

FIG. 9D is an isometric view of a modifiable implant, according to an embodiment.

FIG. 10 is a schematic diagram of a system for modifying an implant, according to an embodiment.

FIG. 11 is a schematic flow diagram of a method of removing an implant, according to an embodiment.

### DETAILED DESCRIPTION

Embodiments disclosed herein are directed to modifiable implants having at least one reactive composite material (“RCM”) associated therewith. Implants have been used in medical fields in a number of ways. Implants can include dental implants, joint replacements, support structure implants for bones, partial bone replacements, jaw implants, pacemakers, drug delivery devices, etc. Subjects for such receiving such implants can include humans and animals alike. Such implants can be used to repair damaged tissue, deliver drugs, or assist biological functions; but require invasive surgeries for implantation, adjustment, or removal. In certain instances, growth or even a reaction to an implant can take place, requiring removal or adjustment of the implant. Such surgeries can be fraught with complications including potential surgery related concerns, difficulty in removing or adjusting an implant that has been implanted in a subject, or potential structural weakness of biological structures such as bones, caused by removal of the implant and the holes or interfaces therein.

Typical structural implants include metal plates, screws, pins, rods, cup and socket, stems, or complete orthopedic prostheses. Implants typically include a set structural flexibility or rigidity. Once implanted, the implant typically has a fixed range of motion only limited by the maximum range of motion built into the implant or the physical limitations of the subject. Thus, if adjustments or removal are required the structure into which the implant is placed must undergo the trauma of removal or adjustment typically associated with orthopedic surgeries, including manually disrupting the surrounding scar and embedding tissue. An implant can include one or more members. For example, an implant can include a first member (e.g., a post or pin) and a second member (e.g., a release member) associated with the first member. The release member can be configured to alter the structural connectivity of the first member with respect to the subject. In implants including a plurality of members; a coating, packaging, adhesive, or other structure for maintaining the plurality of members as one integral unit can be provided. However, as the subject heals or an implant wears out, an altered structural connectivity (e.g., flexibility or connection) between one or more of the members of the implant and the subject may be desired. Such altered structural connectivity can facilitate withdrawal of the implant from subject tissue.

In an embodiment, a system including a modifiable implant can be used to alter the structural connectivity of the implant and facilitate withdrawal of the implant. A system for modifying the structural connectivity of an implant can include an implant having one or more members therein. The one or more members can be structurally connected in such a manner as to allow the one or more members to remain structurally stable in the subject until such time as withdrawal or adjustment of the modifiable implant is desired.

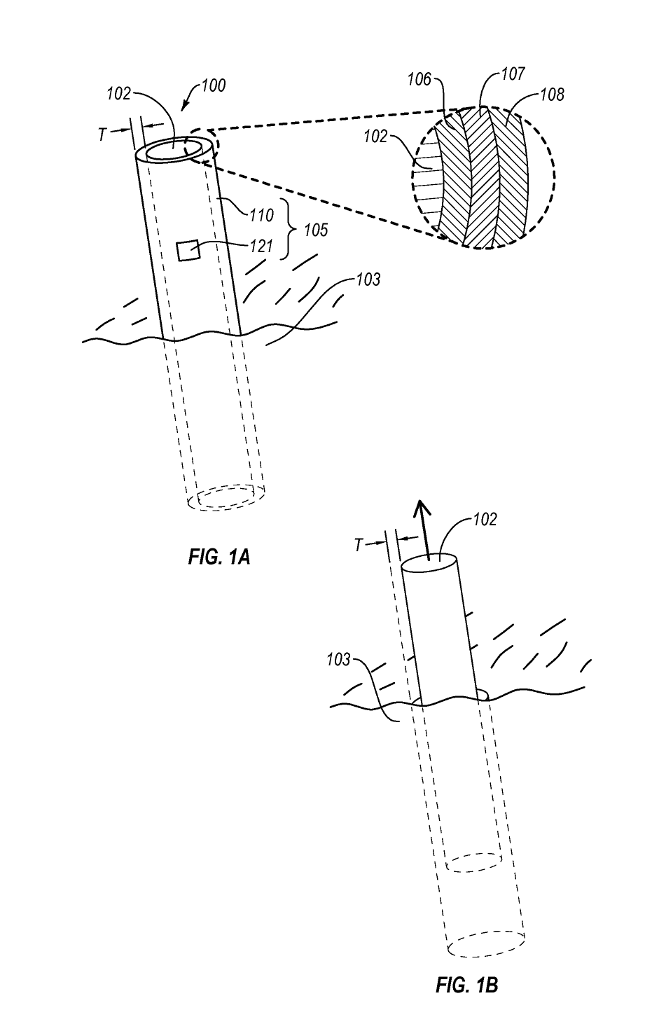

FIGS. 1A and 1B depict a modifiable implant 100 before and after use of a release member associated therewith. The modifiable implant 100 can be at least partially implanted in a subject 103, such as tissue of a human or other animal. The modifiable implant 100 can include one or more bodies 102 and one or more release members 105 configured to promote a change in a structural connectivity of the modifiable implant 100, such as structural connectivity with the subject 103 or the at least one body 102 of the modifiable implant 100. Such structural connectivity can include one or more of the interface between one or more members of the modifiable implant 100, freedom of movement between the members of the modifiable implant 100, the interface between the tissue of the subject 103 and the modifiable implant 100, or the structural rigidity of at least one member of the modifiable implant 100. The at least one body 102 of the modifiable implant 100 can include any suitable implantable material, such as metals (e.g., titanium, surgical steel, stainless steel), ceramics, polymers (e.g., Polyetherketoneketone (PEKK) Polymers), or biological materials (e.g., bone or tissue). The at least one body 102 of the modifiable implant 100 can be configured as or include pins, screws, posts, brackets, drug delivery devices, pacemakers, artificial joints, plates, or other suitable medically implanted structures.

The release member 105 can include a RCM 110, a circuit 121, and a release material associated with the RCM 110. After implantation, the structural connectivity of the modifiable implant 100 can be altered (e.g., in-situ) using the release member 105. The RCM 110 can include one or more layers therein. For example, the RCM 110 can include layers 106-108. The first layer 106 can abut or be adjacent to the body 102, the second layer 107 can be adjacent to the first layer 106, and the third layer 108 can be adjacent to the second layer 107 and remote from the first layer 106. While depicted as having three layers, in an embodiment, the RCM 110 can include less than three layers or more than three layers. As discussed in more detail below, one or more of the layers 106-108 can include one or more of a reactive nanofoil, a release material (e.g., a resistive member, a chemical agent, or a compartment), a protective material, or other suitable materials. The RCM 110 may exhibit a thickness T. The thickness T can include the thickness of the entire release member 105 including the release material. For example, the thickness of the release member 105 extending from the surface of the body 102 outwardly, can be the space between the interface of the modifiable implant 100 with the subject tissue and the at least one body 102 of the modifiable implant 100 including the space occupied by the release material.

The release material can be configured to cause the RCM to at least partially dissociate (e.g., melt, dissolve, or react) upon activation of the release material. Upon triggering the release material, the RCM 110 can be at least partially dissociated (e.g., dissolved, reacted, melted, or otherwise separated). Such dissociation can provide for a change in the structural connectivity of the modifiable implant 100 (e.g., increased compliance of the one or more members to outside forces, altered resilience of the one or more members of the implant, separation of the one or more members of the implant from subject tissue, etc.).

FIG. 1B depicts the modifiable implant 100 of FIG. 1A after activation of the release material. The space formerly occupied by the release member 105 can be empty or substantially vacated after activation of the release material, thereby allowing withdrawal of the remaining portions (e.g., body 102) of the modifiable implant 100 from the subject (e.g., embedding tissue of a human). For example, the at least one body 102 of the modifiable implant 100 can be in the form of a post in which the release member 105 is disposed circumferentially thereabout and having the thickness T. After activation of the release material, the RCM 110 can be substantially completely dissociated (e.g., dissolved, reacted, melted, etc.), leaving a clearance of approximately the thickness T between the subject 103 and one or more portions of the body 102. The body 102 can then be easily withdrawn from the subject, such as by pulling the cylindrical post axially outward from the tissue. Use of the modifiable implant can significantly reduce the duration of medical procedures and the trauma to the tissue surrounding the implants.

The modifiable implant 100 can release from the embedding tissue by substantially completely dissociating the RCM 110, thereby freeing any tissue (e.g., scar tissue, muscle, or bone) formerly connected thereto of the connection to the dissociated RCM 110, and by extension, the implant. The RCM 110 can be configured to allow the body to release from an embedding tissue upon activation of the release material by at least reducing a lateral dimension of the body sufficient to allow withdrawal from an embedding tissue.

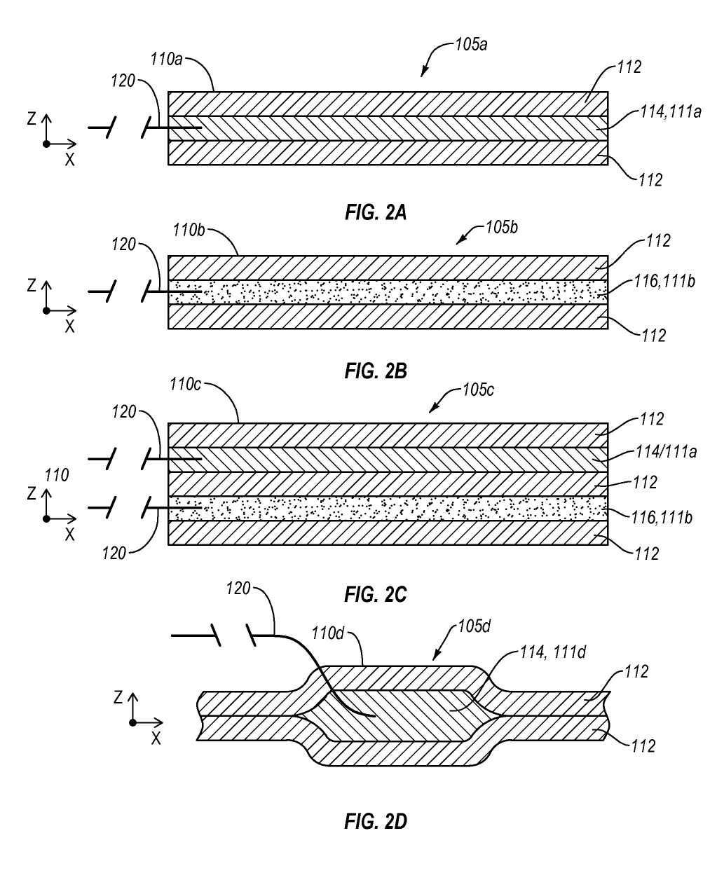

FIGS. 2A-2D are cross sectional views of portions of release members according to embodiments. The release members 105a-105d can include a RCM and one or more release materials. RCMs can include one or more layers therein. The one or more layers can include differing materials in one or more adjacent layers. RCMs can have multiple layers including at least one of reactive foil layers having nanometer or greater thickness that can be referred to herein as “nanofoil”, chemical agents, protective layers, compartments, or resistive members. For example, the RCM can include one or more (e.g., many thousands) layers of reactive nanofoil with a portion of a release material disposed adjacent thereto, such as therebetween. The release member can include a connection to an energy source and the release material is configured to at least partially dissociate the RCM upon activation thereof via the connection to the energy source. The release material can be configured to initiate a chemical or thermal reaction in one or more components (e.g., between two or more components) of the RCM upon activation of the release material. Once initiated (e.g., at one end, or at one corner), the chemical or thermal reaction can self-propagate throughout the RCM, traveling away from the initiation site throughout the entire volume of the RCM.

FIG. 2A is a cross-sectional view of the release member 105a including a portion of RCM 110a having an electrical release material 111a therein. The portion of the RCM 110a includes two layers of reactive nanofoil 112. The reactive nanofoil 112 can include reactive materials such as powders or metals. The powders or metals can be incorporated or impregnated in or incorporated in a binder material such as a polymer, an alloy, a ceramic, or an epoxy. The reactive nanofoil 112 can be produced with a selected thickness via tape casting, CVD deposition, or any other suitable technique. The reactive nanofoil 112 can include alternating layers of materials configured to react with one another or react with an adjacent material (e.g., a portion of a release mechanism). The reactive nanofoil 112 can include discrete portions of one or more materials disposed in a second material in a continuous or discontinuous sheet or pattern. Each individual layer of the reactive nanofoil 112 can be about 1 nm thick or more, such as about 1 nm to about 1 μm, about 5 nm to about 500 nm, about 10 nm to about 200 nm, about 20 nm to about 100 nm, or less than about 500 nm. In an embodiment, individual layers can exhibit the same or differing thicknesses from adjacent layers. In an embodiment, the reactive nanofoil 112 can include substantially only one layer. The reactive nanofoils 112 can include one or more of reactive metals, metal oxides, carbides, nitrides, Al, Ag, Au, B, Ba, Br, C, Ca, Ce, Cl, Cr, Co, Fe, Hf, Mg, Mn, Mo, Nb, Ni, Pd, Rh, Si, Ta, Ti, Th, W, V, Zr, Zn Fe₂O₃, Cu₂O, MoO₃, FeCo, FeCoOₓ, alloys (e.g., monel or Inconel), a metallic glass, a ceramic, or a cermet. For example, reactive nanofoils can include NanoFoil, commercially available from Indium Corporation, comprising alternating nanoscale layers of nickel and aluminum. Other combinations of materials which can be used to form reactive nanofoils are described in U.S. Pat. No. 6,736,942, which is incorporated herein by this reference in its entirety. These reactive nanofoils can include Rh/Si, Ni/Si, Zr/Si, Ni/Al, Ti/Al, Zr/Al, Ti/B, Ti/C, Al/Fe2O3, and Al/Cu2O. Other nanofoil compositions can include any of those described in “Self-Propagating Reactions in Multilayer Materials,” by T. P. Weihs in Handbook of Thin Film Process Technology, 1997, which is incorporated herein by this reference in its entirety. Any of the foregoing materials can be embedded or contained within a thin polymer matrix or substrate to form a reactive nanofoil. In an embodiment, the reactive nanofoil 112 can include a gel or foam (e.g., a hardsetting foam) in one or more layers therein. The gel or foam may be configured to react with or carry one or more reactive nanofoil materials, such as any noted above. Reactive nanofoils 112 can include a thickness of about 1 nm or more such as about 1 nm to about 1 μm, about 5 nm to about 500 nm, about 20 nm to about 300 nm, about 100 nm to about 600 nm, or about 50 nm or more.

A resistive member 114 can be associated with (e.g., disposed between) one or more layers of the reactive nanofoil 112. In an embodiment, the resistive member 114 can include one or more layers of the reactive nanofoil 112 itself (e.g., one or more layers of a Ni/Al or Ti/Al nanofoil). The resistive member 114 can include a material configured to provide resistance to electrical current such as from an electrical connection 120, and thereby heat up upon receiving electrical current. The resistive member 114 can include a material configured to undergo a reaction with an adjacent material (e.g., reactive nanofoil) or self-react upon reaching a temperature effective to initiate such a reaction. For example, the resistive member can include transition metals, refractory metals, alkaline earth metals, alkali metals, or combinations thereof. For example, a suitable resistive member can include aluminum, nickel, iron, copper, zinc, tungsten, or silver.

Upon application of current to the resistive member 114, the resistive member 114 can build-up heat, which can cause one or both of the resistive member 114 or the reactive nanofoil 112 to melt or chemically react. In some embodiments, once resistive member 114 builds up sufficient heat, it can initiate a self-propagating chemical reaction between the materials of reactive nanofoil 1112. Such melting or chemical reaction can result in at least a portion of the RCM 110a dissociating, such as changing from a solid phase material to a liquid or gaseous phase material. In an embodiment, the RCM 110a includes a separate phase-change material (e.g., not one of the components of the nanofoil) which is configured to absorb thermal energy released from the exothermic chemical reaction of the components of reactive nanofoil 112, to increase in temperature, and to undergo a phase change (e.g., from solid to liquid, solid to gas, or solid to liquid to gas, gel to liquid, gel to gas, or foam to liquid). In an embodiment, the phase change material is disposed as one or more layers parallel to layers of reactive nanofoil 112; layers of the phase change material can be interspersed with layers of reactive nanofoil 112, or can be outside reactive nanofoil 112 but proximate to it. In an embodiment, the energy release from the chemical reaction of the components of reactive nanofoil 112, (i.e., M_{NF} gms/area at H_{comb} J/gm) can provide sufficient energy to vaporize M_{PCM} gms/area of phase change material at Hvap J/gm, provided that M_{NF}H_{comb}>M_{PCM}Hᵥₐₚ. In an embodiment, the phase change material comprises a material with low melting or vaporization temperature and/or with low heat of fusion or heat of vaporization. The phase change material may include a plastic (e.g., polyethylene, polycarbonate) or a metal (e.g., sodium, potassium, indium, or gallium). The phase change material may include a gallium, indium, or bismuth alloy (e.g., Indalloy™ available from Indium Corporation).

The material make-up or dimension (e.g., thickness) of the reactive nanofoil 112 can vary depending on one or more of the desired mechanical properties of the RCM 110a or implant on which the RCM 110a is disposed (e.g., structural stability that the RCM 110a provides to the one or more members of the implant in the subject), the type or quantity of release materials (e.g., type or quantity of resistive member or chemical release member) associated therewith, the desired exothermic effect of the reaction of the reactive nanofoil on the surrounding tissue or implant, the number of layers (e.g., of reactive nanofoil, phase change material, protective layers, or release materials) desired in the RCM 110a, or any other suitable criteria. In an embodiment, the RCM in one or more members of an implant can be configured to allow the at least a portion of one or more members to comply with forces placed thereon, such as from embedding tissue resulting in partial bending or crushing of the implant for removal.

As discussed in more detail below, a dimension (e.g., thickness or lateral dimension) of the release material 111a can vary depending one or more of the desired mechanical properties of the RCM 110a or implant on which the RCM 110a is disposed; the type or quantity of release material; the type, properties, thickness, or quantity of layers therein (e.g., reactive nanofoil layers); the desired exothermic effect of the reaction of the reactive nanofoil on the surrounding tissue or implant; or any other suitable criteria. One or more of the dimensions of the release member 105 or RCM 110a can be selected based upon one or more of the above mentioned criteria. For example, the lateral width (e.g., X-axis dimension) of the release member 105 or RCM 110a can be selected to wrap around the circumference of an implant a specified number of times. As another example, the lateral height (e.g., Y-axis dimension) of the RCM 110a can be selected to extend over substantially the entire body of the implant or only a portion thereof. Suitable lateral widths or heights can be 1 mm or more, such as about 1 mm to about 50 cm, about 2 mm to about 25 cm, about 5 mm to about 10 cm, about 50 mm to about 25 mm, about 20 mm to about 5 cm, about 25 mm to about 125 mm, about 25 mm, about 10 mm, 1 mm, about 1 cm, about 2 cm, about 5 cm, about 10 cm, or greater than about 25 mm.

The thickness (e.g., Z-axis dimension) of the release member 105a or RCM 110a can be about 20 nm or more, such as about 20 nm to about 1 mm, about 40 nm to about 500 μm, about 100 nm to about 250 μm, about 500 nm to about 100 μm, about 50 nm to about 500 nm, about 500 nm to about 500 μm, about 25 μm or more, about 200 μm or more, about 100 nm, about 250 nm, about 500 nm, about 5 μm, about 40 μm, about 100 μm, about 200 μm, or about 1 mm or more.

FIG. 2B is a cross-sectional view of the release member 105b including a portion of RCM 110b having release material 111b therein. The portion of the RCM 110b includes two layers of reactive nanofoil 112. Examples of reactive nanofoil materials can include any of those disclosed above. The release material 111b can include at least one chemical release member 116, which can be disposed at one site of reactive nanofoil 112, or between one or more layers of the reactive nanofoil 112. The chemical release member 116 can include a chemical agent or material configured to initiate a self-reaction and/or reaction (e.g., chemical or thermal) with one or more adjacent layers (e.g., nanofoil or chemical agent), upon receiving a stimulus such as electrical current from an electrical connection 120, heat, ultrasonic vibration (e.g., indirectly through an antenna or capacitor triggered by ultrasound signals or directly by about 20 kHz or greater ultrasound signals), microwave radiation, or light (e.g., directly or indirectly via infrared light shone through tissue). In an embodiment, the stimulus can release the chemical agent from a compartment into contact with either another chemical agent or with reactive nanofoil 112. In an embodiment, the stimulus can heat one or more components of the chemical agent so as to initiate combustion between them. The resultant combustion energy can then thermally couple to the reactive nanofoil 112, initiating combustion between the layers of the reactive nanofoil 112. In an embodiment, the self-reacting chemical agent may include a reactive nanofoil. For instance, the RCM 110b may compose a relatively larger portion of one composition of nanofoil coupled in one or more sites to a smaller portion of another reactive nanofoil, serving as the chemical release member 116. The resulting reaction between the at least one chemical release member 116, and in some instances, the reactive nanofoil 112 can result in at least dissociation (e.g., dissolution, degradation, or melting) of the RCM 110b. The at least one chemical release member 116 can include a material configured to undergo a reaction with an adjacent material (e.g., reactive nanofoil) or self-react upon reaching a temperature effective to initiate such a reaction. For example, suitable chemical agents or materials can include one or more of reactive metals, metal oxides, carbides, nitrides, Al, B, Ba, Br, C, Ca, Ce, Cl, Cr, Co, Fe, Hf, Mg, Mn, Mo, Nb, Ni, Pd, Rh, Si, Ta, Ti, Th, W, V, Zr, Zn Fe₂O₃, Cu₂O, MoO₃, FeCo, FeCoOₓ, alloys (e.g., Monel or Inconel), a metallic glass, a ceramic, a cermet, a material configured to react with any of the preceding, or any other chemical compound configured to react with a material in an RCM. Any of the foregoing can be embedded or contained within a polymer. The chemical agent can be in liquid form (e.g., H₂O₂), in powdered form, in solid form (e.g., reactive nanofoil), in gel form, in foam form, incorporated into a binder material or matrix, incorporated into an alloy or a ceramic, or combinations of any of the foregoing.

Upon application of stimulus (e.g., electrical current) to the chemical release member 116, the chemical agents therein can react, which can cause one or both of the chemical agent or the reactive nanofoil 112 to dissociate (e.g., melt or chemically react). Such dissociation can result in at least a portion of the RCM dissociating from the solid form becoming liquid or gaseous.

Multiple stacked layers of RCMs according to any embodiment herein, or single RCMs having multiple layers of any of the components of RCMs disclosed herein—both referred to as RCM stacks—can be used to change a structural configuration of an implant. FIG. 2C is a cross-sectional view of the release member 105c including a portion of an RCM 110c stack having release materials 111a (e.g., resistive member 114) and 111b (e.g., chemical release member 116) therein. The RCM stack 110c can include at least three layers of the reactive nanofoil 112. As depicted, a first layer can be disposed on a first side of a first release material, which can be similar or identical to the release material 111a, including the resistive member 114. A second layer of reactive nanofoil 112 can be positioned one a second side of the release material 111a. A second release material can be positioned adjacent to the second layer of reactive nanofoil. For example, the second release material can be configured similar or identical to release material 111b, including the chemical release member 116. A first side of the release material 111b can be positioned adjacent to the second layer of the reactive nanofoil 112, such as one the side opposite the release material 111a. The resistive member 114 and/or chemical release member 116 can include any of those respective materials disclosed above. A third layer of reactive nanofoil 112 can be positioned on the second side of the release material 111b. In an embodiment, the RCM can include more than one release material therein, such as about two to about 10 release materials, about two release materials, about three release materials, or about 5 release materials or more.

In an embodiment, RCMs can be layered over one another to form a RCM stack having a plurality of RCM layers therein. The number of the RCM layers can be increased or decreased to produce a selected thickness of the RCM stack. A respective RCM stack can include more than one of any of the RCMs describe herein. For example, an RCM stack can include a plurality of layers each including the RCM 110a shown in FIG. 2A. In an embodiment, the RCM stack 110c can include a plurality of layers including one or more of the RCMs 110a shown in FIG. 2A, the RCM 110b shown in FIG. 2B, or other configurations including one or more of any of the RCM layers disclosed. The thickness of the RCM stack 110c can vary depending on the desired mechanical strength of the RCM stack, the materials in the RCMs therein, the implant type, the desired clearance of the implant upon removal, or other suitable criteria. An adhesive layer (not shown) can be present between any of the adjacent layers of the RCM stack 110c. The thickness of an RCM stack can be about 50 nm or more such as about 50 nm to about 200 μm, about 100 nm to about 100 μm, about 250 nm to about 50 μm, about 500 nm to about 1 μm, about 50 nm to about 500 nm, about 500 nm to about 1 μm, about 1 μm to about 150 μm, about 200 μm or less, about 100 nm, about 250 nm, about 500 nm, or less than 1 mm. RCM stacks can be used interchangeably with the RCMs in any of the embodiments herein.

In an embodiment, multiple layers of reactive nanofoil can be overlaid upon each other. Each layer can be different to an adjacent layer, such that the adjacent layers are configured to react with one another upon receiving a sufficient stimulus, such as from a release member or other stimulus source. In an embodiment, each layer can be substantially similar or identical to each adjacent layer.

FIG. 2D is a cross-sectional view of a release member 105d including a portion of RCM 110d having material 111d therein. In an embodiment, release material can be positioned across an entire lateral dimension (e.g., in the X and Y directions) of an RCM or can be disposed in discrete portions an RCM extending less than the entire lateral dimension of an RCM, such as in a band, a pocket, or pattern (e.g., continuous or discontinuous patterns). As shown in FIG. 2D, two layers of the reactive nanofoil 112 can be substantially in contact with each other throughout an RCM. At one or more intermediate points therein, the RCM 110d can include one or more release materials therein. As shown, the release material 111d can be configured as resistive member 114 and can be disposed within a discrete lateral portion of the RCM 110d such that activation of the release material 111d can affect regions of the RCM adjacent to the release material 111d. This discrete lateral portion can be positioned adjacent to a portion of the implant in which increased compliance (e.g., increased flexibility or rotation) is desired after implantation. Upon activation of the release member 105d, portions of the RCM 110d adjacent to the release material 111d can dissociate leaving other more distant portions of the RCM 110d substantially unaffected. Selected portions of the RCM 110d (e.g., portion surrounding the joint of an implant) can be selectively removed or otherwise dissociated via such an embodiment.

In an embodiment, a release material can be disposed within a discrete lateral portion of the RCM. The reactive nanofoil can be configured to dissociate across the entire lateral dimensions thereof responsive to a reaction with or caused by the activated release material in only a portion of the RCM. Thus, in an embodiment, only a portion of the RCM can include the release material capable of causing substantially the entire RCM to at least partially dissociate. For example, an RCM can include a release material associated therewith in a checkerboard pattern or a linear pattern.

In an embodiment, at least a portion of the release material can occupy about 100% of the lateral area of the RCM. In an embodiment, at least a portion of the release material can occupy less than 100% of the lateral area of the RCM, such as 90% or less of the lateral area, about 90% to about 5%, about 75% to about 25%, about 60% to about 40%, about 50% to about 10%, about 20% to about 5%, about 10%, about 25%, or about 50% of the lateral surface area of the RCM. In an embodiment, at least a portion of the release material can extend across an entire a lateral dimension of the RCM. For example, the release material can extend horizontally across (e.g., in the X-axis direction) an RCM in a small band occupying about 10% of the lateral height (e.g., the Y-axis direction) of the RCM. In an embodiment, at least a portion of the release material can occupy a discrete pocket, pattern (e.g., continuous or discontinuous pattern), or isolated lateral portion of the RCM. Such embodiments can promote structural rigidity yet allow change structural connectivity via dissociation of only a small portion of the total RCM.

In an embodiment, the RCM can include one or more protective layers configured to reduce or eliminate the effects of the release material or reactions of the RCM therewith from penetrating into adjacent tissue or the implant. For example, a protective layer can be configured to protect adjacent tissue from the chemical or thermal effects (e.g., increased temperature) of a reaction between the reactive nanofoil and the release material. In such an embodiment, the protective layer can include an endothermic reactant configured to react with one or more of the reactive nanofoil, the release material, or the products of a reaction therebetween to cause an endothermic or neutralizing reaction therewith. The endothermic or neutralizing reaction can limit the extent of heat from an exothermic reaction or extent of damaging chemical reactants (e.g., acidic or basic chemical species) caused by activation of the release material. Such an embodiment can provide an enthalpy of reaction with relation to the surrounding tissue and/or implant as near to zero as possible.

In an embodiment, the protective layer can include one or more chemical reactants configured to react with one or more of the reactive nanofoil, the release material, or the products of a reaction therebetween to neutralize the chemical components thereof to at least limit toxic or corrosive chemicals from damaging subject tissue or the implant. The protective layer can include one or more compounds or molecules embedded within a substrate such as a polymer, epoxy, ceramic, metal alloy, or cermet. Suitable endothermic reactants can include inorganic or organic reactants such as hydrated barium hydroxide, alumina trihydrate, ammonium chloride, nitrates, thiocyanate, thionyl chloride and cobalt(II) sulfate heptahydrate, or any other suitable reactant configured to react with the chemical agent, reactive nanofoil, or reaction products thereof.

The protective layer can be configured such that the protective layer also dissociates upon activation of the release material. The thickness or material of the protective layer can be selected based upon one or more of the type of the release material or reactive nanofoil, the size (e.g., thickness) of the release material or reactive nanofoil, the heat expected to be generated by the release material, the type material of the implant, or the type of tissue in which the implant is deployed. In an embodiment, the thickness of the protective layer can be sufficient to provide enough of an endothermic reactant to react with the release material and/or reactive nanofoil to substantially limit or eliminate the exothermic effects therefrom from damaging surrounding tissue. The thickness of the protective layer can also be selected to provide substantially only enough material in the protective layer to react with one or more of the release material, the reactive nanofoil or products thereof, such that the protective layer is substantially dissociated (e.g., dissolved or exhausted) upon use thereof.

In an embodiment, an RCM can include the protective layer between the reactive nanofoil and the tissue of a subject. The protective layer can exhibit a thickness sufficient to limit the effects of the release material on the surrounding tissue due to use of the release material, such as the resistive member heating up, reaction of the release material and/or reactive nanofoil, or any other release material effects.

In an embodiment, the protective layer can be disposed adjacent to the entire release member, such as one or more of between the release member and the subject or between the release member and the body. In an embodiment, the protective layer can be disposed adjacent to the release material. For example, in an embodiment where the release material extends across only portion of a lateral dimension of the RCM, the protective layer can similarly extend over only a portion of a lateral dimension of the RCM, including only over the same portions as the release material described above. In an embodiment, protective layer can be disposed substantially parallel to or blanketing the portions of the RCM having the release material therein. In an embodiment, protective layer can be disposed substantially parallel or blanketing the portions of the RCM having the release material therein and extend slightly past such portions by a distance to ensure limitation of negative effects from use of the release material on surrounding environments. In an embodiment, the protective layer can cover (e.g., overlap) a larger lateral dimension of the RCM containing the release material by 2% of the lateral dimension of the release material or more, such as a 5% to about 50% larger lateral dimension, or covering a 10% larger lateral dimension.

The RCMs described herein can be used to provide structural connectivity (e.g., connection, rigidity, etc.) between one or more members of an implant, or the implant and the subject. Any of the RCMs described herein can be disposed in or on an implant to provide a modifiable structural connectivity to the implant, such as allowing increased motion of one member of an implant with respect the subject in which the implant is connected to (e.g., rotation, axial movement, flexibility, lateral movement, bending, sliding, separation, etc.). An RCM can be disposed across or around the one or more bodies of an implant, whereby the release material causes the RCM to at least partially dissociate, thereby modifying or allowing the modification of the structural connectivity of the body of the implant.

In an embodiment, the reactive nanofoil of an RCM can be configured to undergo self-reaction between the constituents thereof responsive to a stimulus (e.g., electrical, thermal, acoustic, etc.) such that a release material is not required to at least partially dissociate the RCM. In such embodiments, an operable connection between the stimulus source (e.g., electrical connection to a capacitor) and one or more layers of reactive nanofoil can be included in the release member to initiate reaction of the RCM. In such embodiments, the RCM can include substantially only reactive nanofoil layers of one or more compositions.

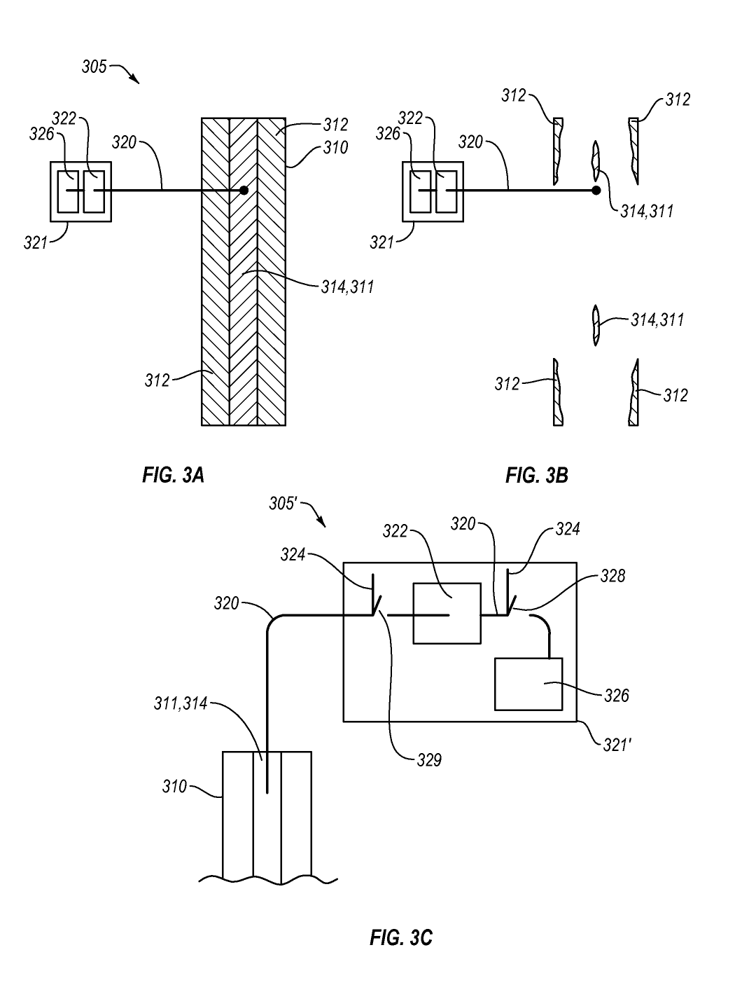

FIGS. 3A and 3B are schematic cross-sectional views of a release member 305 of RCM 310 before and after use, according to an embodiment. FIG. 3A depicts the release member 305 including the RCM 310 and a release material 311 therein prior to activation of the release material 311. In an embodiment, the release material 311 can include a resistive member 314 operably coupled to a circuit 321 by an electrical connection 320 therebetween. The circuit 321 can include a battery 326 operably coupled to a capacitor 322. In an embodiment, the capacitor 322 can be configured to be charged by the battery 326 and discharged through the resistive member 314 operably coupled to the capacitor 322. In an embodiment, the capacitor 322 can be configured to be charged by ultrasonic vibrations or infrared light directed to an antenna, resonator, or the battery 326.

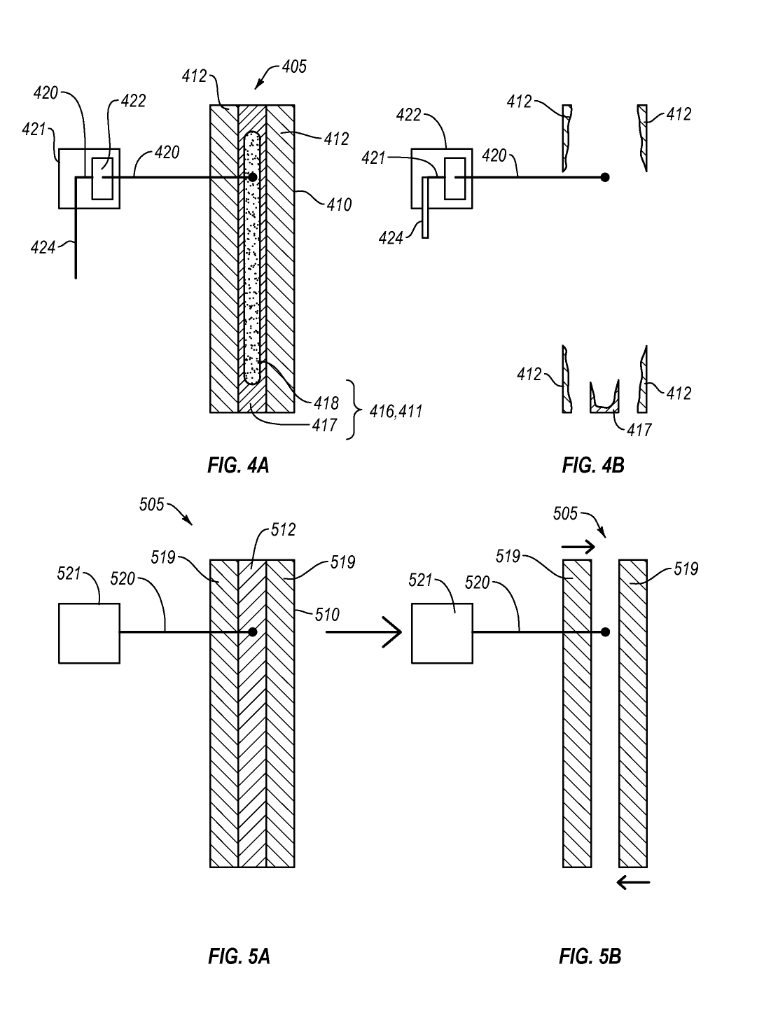

In an embodiment, the capacitor 322 can be configured to be charged via electromagnetic energy (e.g., radio frequency energy) directed to an antenna (FIGS. 4A and 4B) configured to harvest electromagnetic radiation and discharged through the resistive member 314 operably coupled to the capacitor 322. In an embodiment, the circuit 321 can include a resonator (not shown), such as a resonator configured to receive a specific frequency RF signal (e.g., narrow-band resonator).

The RCM 310 can be configured identical or similar to any of the RCMs disclosed herein, such as RCMs 110a-110d. The resistive member 314 can be similar or identical to the resistive member 114. For example, the resistive member 314 can be disposed between one or more layers of the reactive nanofoil 312. For example, the resistive member 314 can be disposed at one site, an edge, or a corner of the reactive nanofoil in the RCM 310. The resistive member 314 can include a material configured to provide resistance to electrical current such as from the electrical connection 320, and thereby heat-up upon receiving electrical current. The resistive member 314 can include a material configured to undergo a reaction with an adjacent material (e.g., reactive nanofoil) or self-react upon reaching a temperature effective to initiate such a reaction.

The battery 326 can be configured such that it does not add unsatisfactory bulk or volume to the implant, release member 305, or RCM 310. For example, suitable batteries 326 can include one or more of a thin film battery, a button cell, a zinc-air cell (e.g., using oxygen from the water in surrounding tissues or fluids), or suitable other miniaturized batteries. A suitable thin film battery can include a flexible thin film lithium-ion battery, such as the LiTe*STAR™ thin-film rechargeable battery or Thinergy® battery by Infinite Power Solutions, or equivalents thereof. The battery 326 can be configured to deliver 0.1 mV or more, such as about 0.1 mV to about 20 V, about 0.5 mV to about 5 V, about 1V to about 1000V, about 0.5 V, about 1 V, about 2 V, or about 10 V or less. The battery 326 can be configured to deliver 0.1 mA or more, such as about 0.1 mA to about 1 A, about 0.2 mA to about 0.5 mA, or about 1 A. The battery 326 can be operably coupled to the capacitor 322 via an electrical connection sufficient to allow the capacitor to charge when a closed circuit is formed therebetween. The battery 326 can be connected to the capacitor 322 via a voltage enhancing circuit so as to charge the capacitor to a higher voltage than that of the battery.

The capacitor 322 can include an implantable capacitor having a sufficiently small size to be associated with one or more portions of the modifiable implant. For example, suitable capacitors can include miniaturized ceramic or electrolytic capacitors such as those made and sold under the TAZ series name by AVX Corporation of Fountain Inn, South Carolina. The capacitor size or capacitance can be selected based upon one or more of the size of the implant, the size of the RCM 310, the type of RCM 310, the type of resistive member 314, the type of chemical agent or material, or combinations thereof. The capacitor 322 can be configured with a 1 nF capacitance or greater, such as about 0.001 μF to about 1000 μF, about 0.01 μF to about 100 μF, or about 0.1 μF to about 10 μF. The capacitor 322 can be configured to deliver a voltage of 0.1 mV or more, such as about 0.1 mV to about 20 V, about 0.5 mV to about 5 V, about 0.5 V, about 1 V, about 2 V, or about 10 V or less. The capacitor 322 can be configured to discharge through the resistive member 314.

The capacitor 322 can be connected to the resistive member 314 by one or more of the electrical connections 320. The one or more of the electrical connections 320 can be of sufficient length to allow the capacitor 322 to be remote from the resistive member 314 or the RCM 310, such as in tissue of a subject, in or on a member of the implant, or external to the subject. For example, the electrical connection 320 can be at least about 100 μm long, such as about 1 mm to about 20 cm, about 5 mm to about 10 cm, about 10 mm to about 2 cm, less than 10 cm, or greater than about 10 mm long. The one or more of the electrical connections 320 can be a wire of any suitable material configured to efficiently conduct electrical current therethrough. The wire can include an insulating material over at least a portion thereof. The insulating material can prevent voltage loss to the external environment such as the subject tissue or implant. In an embodiment, the electrical connection 320 can include a copper wire connected to the resistive member 314 extending away from the RCM 310 to the capacitor 322 remote from the RCM 310. The wire can be coated with an insulator thereabout except for portions thereof in contact with or within the resistive member 314 or the capacitor 322.

As shown in FIG. 3A, the resistive member 314 can be positioned substantially completely throughout the RCM 310 (e.g., extends across substantially the entire lateral dimensions of the RCM 310). In such embodiments, the release member 305 can include one or more of the electrical connections 320 therein, to ensure substantially complete dissociation of the RCM 310. In such embodiments, substantially the entire RCM 310 can be dissociated (e.g., dissolved, reacted, or melted). For example, the RCM 310 can be melted or reacted (e.g., thermally or chemically) at least partially from a solid state to a liquid state, at least partially from a solid state to a gaseous state, at least partially from a gel to a liquid, at least partially from a gel to a gas, at least partially from a foam to a liquid, or combinations thereof.

FIG. 3B is a schematic diagram of the release member 305 of FIG. 3A after the release material 311 has be activated. As shown in FIG. 3B, upon activation of the release material 311 in the release member 305, such as by application of voltage from the capacitor 322, the RCM 310 can be at least partially dissociated. One or more portions of the RCM 310 can be substantially completely dissociated, such as by chemical or thermal reaction. Notwithstanding dissociation of one or more portions of the RCM 310, some vestiges of one or more portions of the RCM 310 can remain unreacted or in solid form after the release member 305 is activated. Such remaining portions should not interfere with the modified structural connectivity of the implant. The electrical connection 320 can remain after dissociation of the RCM 310, however, in an embodiment one or more portions of the electrical connection 320 can be configured to at least partially dissociate with the RCM 310.

In an embodiment, the resistive member 314 can be positioned at a discrete intermediate point of the RCM 310 substantially perpendicularly therethrough (e.g., substantially perpendicular to the direction of the layers of the RCM 310). In an embodiment, the resistive member 314 can be positioned or extend laterally therethrough (e.g., substantially parallel to the direction of the layers of the RCM 310), at a discrete location (e.g., a substantially spherical or polygonal body) therein, or any other position (e.g., continuous or discontinuous patterns) suitable to allow the resistive member 314 to react with one or more components of the RCM 310. In an embodiment, only the portion of the RCM 310 adjacent to the resistive member 314 can react therewith or otherwise dissociate (e.g., melt). In such embodiments, a gap in the RCM 310 can be observed in the immediate area the resistive member 314 occupied prior to activation.

FIG. 3C is a schematic diagram of a release member 305′ according to an embodiment. In an embodiment, the release member 305′ can include the RCM 310, a circuit 321′, the electrical connection 320, and a release material 311′. The circuit 321′ can include the capacitor 322 and one or more of the batteries 326 operably coupled thereto via the electrical connection 320. The capacitor 322 can be configured to be charged (e.g., slowly charged) via the battery 326 and discharged (e.g., rapidly discharged) through the chemical release member or resistive member operably coupled to the capacitor 322. Thus, in an embodiment the release member 305′ can be triggered by a small electrical pulse. The capacitor 322 and the electrical connection 320 can be similar or identical to any capacitor or electrical connection described herein. The battery 326 can be configured similarly or identical to any battery disclosed herein.

The circuit 321′ can further include an electrical switch 328 or gate between the battery 326 and the capacitor 322. The electrical switch 328 can be operably coupled to an antenna 324, whereby the antenna 324 can be configured to receive a specific stimulus, such as a specific frequency or wavelength of electromagnetic radiation, ultrasonic vibrations, or infrared light. In an embodiment, the antenna 424 is coupled to a narrow-band resonator, configured to respond to a specific stimulus (e.g., specific type or frequency). For example, the electrical switch 328 can be configured to close upon receipt of an electrical stimulus from the antenna 324 responsive to the appropriate frequency or wavelength of electromagnetic radiation. The electrical switch 328 can include a MEMS switch, such as an RF switch or a microwave switch by way of example. Once the electrical switch 328 closes, the battery 326 can charge the capacitor 322, and the capacitor 322 can discharge. In an embodiment, an electrical switch 329 can be located between the capacitor 322 and the RCM 310 or release material associated therewith, such that the capacitor 322 can only be discharged into the RCM 310 upon receipt of the appropriate frequency or wavelength of electromagnetic radiation. The electrical switch 329 can be similar or identical to the electrical switch 328. In an embodiment, the release member 305′ can include one or more of the electrical switches 328 or 329 described above. For example, the circuit 321′ can include the electrical switch 328 coupled to an antenna 324 and configured to receive a first radio frequency (e.g., includes a first narrow-band resonator); and the electrical switch 329 can be coupled to an additional antenna 324 and configured to receive a second radio frequency (e.g., includes a second narrow-band resonator tuned to a different frequency than the first narrow band resonator). In such an embodiment, the capacitor 322 can only be charged and discharged into the RCM 310 upon receipt of both radio frequencies. In an embodiment, a plurality of electrical switches 328 or 329 and associated antennas 324 can be configured to operate on the same stimulus or differing stimuli. In an embodiment, the circuit 321′ can be disposed in or on an implant, such as in or on a member of the implant, in or on the RCM, or combinations thereof. Suitable radio frequency radiation can include those used for radio, telephone, wireless telephone, or other suitable radio frequencies. In an embodiment, portions of the circuit 321′ of the release member 305′ (e.g., antennas or resonators) can be configured to receive an encrypted or narrow-band radio signal to limit the chance of accidental activation of the release member or release materials therein. An antenna associated with the circuit 321′ can be configured to respond to a different stimulus (e.g., different type, frequency, polarity, or wavelength) and be similarly activated using the different stimuli.

The antenna 324 can include a pin antenna, monopole antenna, a dipole antenna, a resonator (as explained in more detail below) or any other structure capable of harvesting electromagnetic radiation (e.g., radio frequency radiation), sonic vibrations, or light to produce an electrical charge or current. The antenna 324 can extend away from the capacitor 322, electrical connection 320, circuit 321′, or RCM 310. The antenna 324 can be at least partially integrated into the structure of one or more of the circuit 321′, capacitor 322, electrical connection 320, or RCM 310. The antenna 324 can be tuned to a particular frequency, polarity, or wavelength, such that the release material 311′ is not unintentionally activated. The circuit 321′ can also include one or more rectifiers (not shown) between the antenna 324 and the switches 328 or 329 to convert alternating current to direct current. Thus, in an embodiment the release member 305′ can be triggered by a small electrical pulse.

In an embodiment, the release member 305′ can include a circuit having one or more resonators therein, such as in addition to an antenna. The one or more resonators can be configured to receive narrow-band radiofrequency radiation. The one or more resonators can collect the narrow-band radio frequency radiation and convert the radiofrequency radiation into electrical current or generate a higher voltage or current than is received therein. Suitable resonators can include MEMS devices such as MEMS resonators or miniaturized RLC circuits.

FIGS. 4A and 4B are schematic diagrams of release member 405 including release material 411 and cross-sectional views of RCM 410 before and after use, according to an embodiment. FIG. 4A depicts the release member 405 including the RCM 410 and the release material 411 therein. The release material 411 includes at least one chemical release member 416 operably coupled to a circuit 421 by electrical connection 420. The circuit 421 can include a capacitor 422 and an antenna 424 operably coupled to the capacitor 422 by electrical connection 420. The capacitor 422 or electrical connection 420 can be identical or similar to the respective capacitor 322 or electrical connection 320. In an embodiment, the capacitor 422 can be configured to be charged (e.g., slowly charged) by the antenna 424 and discharged (e.g., rapidly discharged) through the chemical release member 426 operably coupled to the capacitor 422. In an embodiment (not shown), the circuit 421 can include a battery configured to charge the capacitor 422. Thus, in an embodiment the release member can be triggered by a small electrical pulse.

The antenna 424 or aspect thereof can be similar or identical to any antenna or aspect thereof disclosed herein, including any rectifier or resonator associated therewith. The circuit 421 can also include one or more rectifiers (not shown) between the antenna 424 and the capacitor 422 to convert alternating current to direct current.

The RCM 410 can be configured identical or similar to any of the RCMs disclosed herein, such as any of RCMs 110a-110d, or 310. The at least one chemical release member 416 can be similar or identical to the chemical release member 116. For example, the chemical release member 416 can be disposed between one or more layers of the reactive nanofoil 412 in the RCM 410.

The RCM 410 can include a first layer of reactive nanofoil 412, a second layer of reactive nanofoil 412, and a chemical release member 416 therebetween. In an embodiment, the chemical release member 416 can be configured similar or identical to the chemical release member 116. In an embodiment, the chemical release member 416 can include a compartment 417 including a chemical agent 418 therein. The compartment 417 can include one or more walls completely enclosing the chemical agent 418 therein. The compartment 417 can include a material configured to remain stable until receipt of a stimulus (e.g., electrical current from the electrical connection 420). The material forming the compartment 417 can be configured to melt, react, or otherwise interact with the chemical agent 418 upon receipt of a stimulus, sufficient to at least partially dissociate the RCM 410 adjacent thereto. In an embodiment, only a portion of the compartment 417 can be configured to respond to receipt of the stimulus, while a second (e.g., larger) portion remains inert. Upon reaction of the chemical agent 418 (e.g., with the material of the compartment 417 or melting the material of the compartment 417), one or more of the chemical agent 418, a product of the reaction of the chemical agent 418, the material of the compartment 417, or a product of the reaction of the material of the compartment 417 can react with the reactive nanofoil 412 adjacent thereto, to at least partially dissociate the RCM 410.

In an embodiment, the material of the compartment 417 can include inert or reactive components therein. The material of the compartment 417 an include transition metals, alkaline earth metals, alkali metals, organic compounds (e.g., polymers), inorganic compounds, ceramics, or other suitable compounds. In an embodiment, the stimulus can release the chemical agent 418 from the compartment 417 coming into contact with either another chemical agent or with reactive nanofoil 412. In an embodiment the stimulus can heat one or more components of the chemical agent 418 so as to initiate combustion between them. The resultant combustion energy can then thermally couple to reactive nanofoil 412, initiating combustion between its layers. In an embodiment, the self-reacting chemical agent 418 may include a reactive nanofoil such as, for instance, the RCM may compose a relatively larger portion of one composition of nanofoil coupled in one or more sites to a smaller portion of another reactive nanofoil, serving as chemical release member 416. The chemical agent 418 can include a chemical or chemical compound configured to react upon stimulation, such as via electrical current from the electrical connection 420 or sonic vibration (e.g., ultrasound). The chemical agent 418 can include one or more of reactive metals, metal oxides, carbides, nitrides, Al, B, Ba, Br, C, Ca, Ce, Cl, Cr, Co, Fe, Hf, Mg, Mn, Mo, Nb, Ni, Pd, Pt, Rh, Si, Ta, Ti, Th, W, V, Zr, Zn Fe₂O₃, Cu₂O, MoO₃, FeCo, FeCoOₓ, alloys (e.g., monel or Inconel), a metallic glass, a ceramic, a cermet, or any other chemical compound configured to react with one or more materials in an RCM (e.g., reactive nanofoil layer). The chemical agent 418 can be in liquid form (e.g., H₂O₂), in powdered form, in solid form (e.g., a reactive nanofoil), incorporated into a binder material or matrix, or incorporated into an alloy or a ceramic. Any of the foregoing can be embedded or contained within a thin polymer matrix or substrate.

As shown in FIG. 4A, at least one chemical release member 416 can be positioned between a plurality of layers of reactive nanofoil 412. In an embodiment, one or more of the release member 405, the RCM 410, or chemical release member 416 can be positioned or extend substantially perpendicularly or laterally at least partially about a body, at a discrete location therein, or any other position suitable to allow the chemical release member 416 to react with one or more components of the RCM 410. In an embodiment, the RCM 410 can be configured such that the chemical release member 416 extends about substantially the entire lateral dimensions of the RCM 410. In such embodiments, the release material 411 can include one or more electrical connections 420 operably coupled thereto, to ensure satisfactory dissociation of the RCM 410. In such embodiments, substantially the entire RCM 410 can be dissociated (e.g., dissolved, chemically reacted, or melted). For example, the RCM 410 can be melted or reacted at least partially from a solid state to a liquid state, at least partially from a solid state to a gaseous state, or combinations thereof.

In an embodiment, the chemical release member 416 can be positioned with respect to the RCM 410 similarly or identical to the positions described with respect to resistive member 314. In an embodiment, only the portion of the RCM 410 adjacent to the chemical release member 416 can react therewith or otherwise dissociate (e.g., melt). In such embodiments, a gap in the RCM 410 can be observed in the immediate area the chemical release member 416 occupied prior to activation.

As shown in FIG. 4B, upon activation of the release material 411, such as by application of voltage from the capacitor 422, the RCM 410 can be at least partially dissociated. One or more portions of the RCM 410 can be substantially completely dissociated, such as by chemical or thermal reaction. Notwithstanding dissociation of one or more portions of the RCM 410, some vestiges of one or more portions of the RCM 410 can remain unreacted or in solid form after the release material 411 is activated. Such remaining portions should not interfere with the modified structural connectivity of the implant. The electrical connection 420 can remain after dissociation of the RCM. However, in an embodiment, the portion of the electrical connection 420 in contact with the chemical release member 416 can also at least partially dissociate.

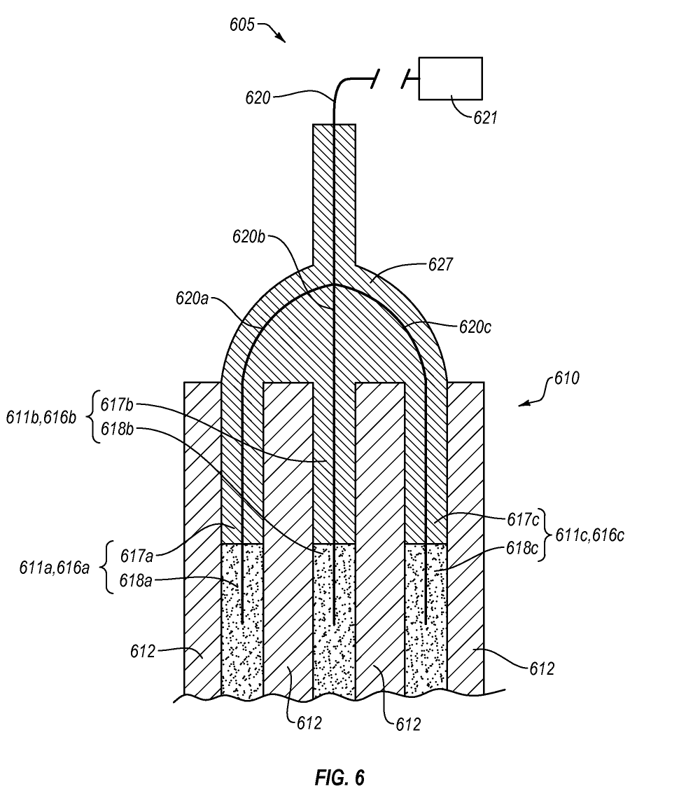

FIGS. 5A and 5B are schematic diagrams of release member 505 including release material 511 and cross-sectional views of RCM 510 before and after use, according to an embodiment. In an embodiment, one or more portions of the RCM 510 can remain intact after activation of the release material 511 associated therewith. In an embodiment, the RCM 510 can include one or more layers therein. The one or more layers can include one or more of reactive nanofoil, at least a portion of a release material, or a protective layer. The RCM 510 can include a reactive nanofoil 512 disposed between a plurality of protective layers 519. The reactive nanofoil 512 or protective layer 519 can be configured identical or substantially similar to any reactive nanofoil or protective layer, disclosed herein. The reactive nanofoil can be operably connected to a circuit 521. The release member 505 can include an electrical connection 520 coupled to the reactive nanofoil 512 and circuit 521. The circuit 521 can be configured to deliver a stimulus (e.g., electrical current) to the reactive nanofoil 512 effective to cause the reactive nanofoil 512 to at least partially dissociate. In such an embodiment, the reactive nanofoil 512 can be considered the release material 511. The reactive nanofoil 512 can be used as a release material in any of the embodiments herein. The circuit 521 can be similar or identical to any circuit disclosed herein.

FIG. 5B shows the release member 505 from FIG. 5A after the reactive nanofoil 512 has been activated. In an embodiment, the protective layers 519 can be configured to remain at least partially intact after activation of the release member 505, such as by one or more of thickness, composition, number of protective layers, or position of protective layers. In an embodiment, the protective layers 519 can be configured remain substantially completely intact after activation of the release member 505 and at least partially dissociation of the reactive nanofoil 512. The overall thickness of the RCM 510 can be reduced by the absence of one or more layers of reactive nanofoil 512, thereby reducing at least one overall dimension of the release member 505 and implant. The absence of one or more reactive nanofoil layers 512 can allow the remaining protective layers to move with respect to each other responsive to forces placed thereon, such as pulling or crushing forces. Such a configuration can allow for withdrawal of an implant from a subject without dissociating the entire RCM 510. Such embodiments can also reduce potential for damage to surrounding tissue or the implant by including protective layers 519 having one or more of an endothermic reactant, a neutralizing reactant, or a reaction rate controlling reactant therein.

In an embodiment, the protective layers 519 can be at least partially dissociated, such that one or more portions thereof are not present after activation of the at least one release material (e.g., reactive nanofoil). For example, the protective layers 519 can be configured to at least partially change from a solid phase material to a liquid or gas phase material.

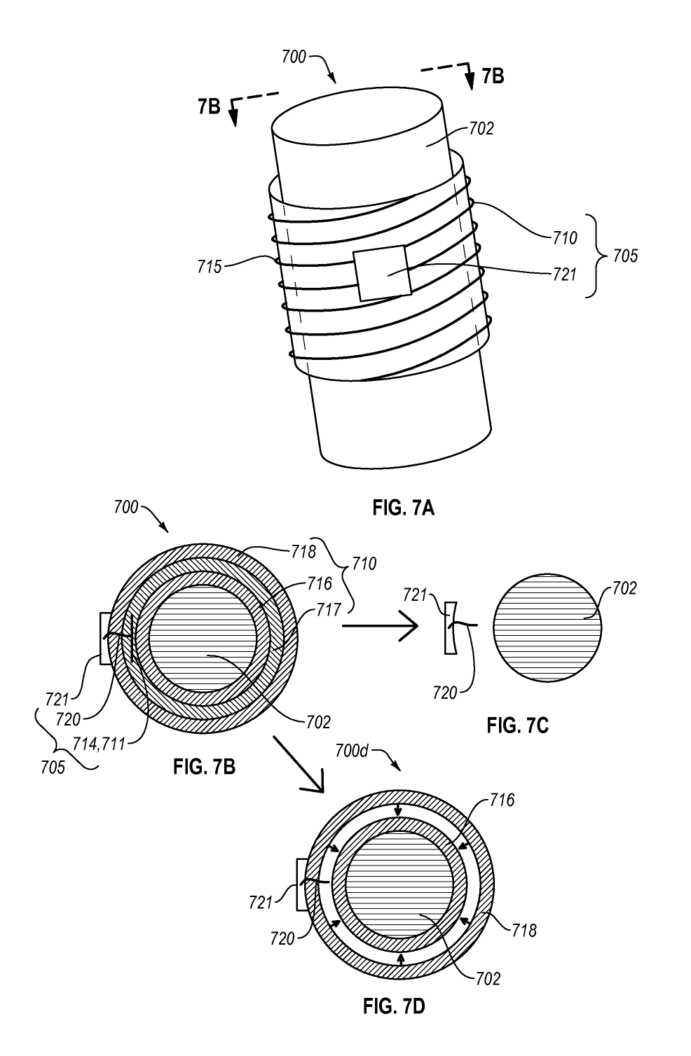

FIG. 6 is a schematic cross-sectional view of an embodiment of a release member 605 including RCM 610 having release materials 611a-611c, with the RCM 610 shown before use. The RCM 610 can include a plurality of reactive nanofoil layers 612 and release material 611a-611c therebetween. The release material 611a-611c can include at least one chemical release member 616a-616c, at least one circuit 621, and one or more electrical connections 620 therebetween. The circuit 621 can be configured identically or similarly to any circuit disclosed herein. The release material 611a-611c or the circuit 621 can include one or more electrical switches (not shown) similar or identical to switches 328 or 329.

The circuit 621 can include one or more of a capacitor (not shown), electrical connection 620, resonator, or antenna (not shown), which each can be identical or similar to a respective capacitor 322 or 422, electrical connection 320 or 420, resonator, or antenna 324 or 424, disclosed herein. The RCM 610 or any sub-components thereof can be configured identical or similar to any of the RCMs or sub-components thereof disclosed herein, such as RCMs 110a-110d. One or more of the chemical release members 616a-616c or portions thereof can be similar or identical to the chemical release member 116 or 416 or portions thereof. Each of the at least one chemical release members 616a-616c can be disposed between one or more layers of the reactive nanofoil 612 in the RCM 610. Each chemical release member 616a-616c can include a chemical agent 618 or material configured to react or initiate a reaction responsive to electrical current such as from the electrical connection 620. One or more of the chemical release members 616a-616c can be similar or identical to one or more of the adjacent chemical release member 616a-616c. One or more of the chemical release members 616a-616c can be different from one or more of the adjacent chemical release member 616a-616c, such as being located in a different lateral portion of the RCM 610 or including a different chemical agent or compartment material therein. For example, the chemical agent 618b can be configured to exothermically react with the reactive nanofoil 612 adjacent thereto, and the chemical agents 618a and 618c can be configured to endothermically react with one or more of the reactive nanofoil 612, the product of the reaction between the reactive nanofoil 612 and the chemical agent 618b adjacent thereto, sufficient to limit negative effects of the exothermic reaction of chemical agent 618b on the surrounding tissue or implant.

The circuit 621 can be operably coupled to the release materials 611a-611c by one or more electrical connections 620. The electrical connection 620 can extend to or through a manifold 627. In the manifold 627, the electrical connection 620 can split into a plurality of branches, such as electrical connections 620a-620c. In an embodiment, the electrical connection 620 can be a harness including a plurality of wires, such as electrical connections 620a-620c. In an embodiment, each electrical connection 620a-620c can be operably connected to least one capacitor, such as each to the same or a different at least one capacitor.

Each electrical connection 620a-620c can be operably connected to a respective resistive member, a material of a compartment 617a-617c, or a chemical agent 618a-618c. As shown, the electrical connection 620a can branch from electrical connection 620 in the manifold 627, extend into the compartment 617a, and into the chemical agent 618a therein, thereby forming a chemical release member 616a. The electrical connection 620b can branch from electrical connection 620 in the manifold 627, extend into the compartment 617b, and into the chemical agent 618b therein, thereby forming a chemical release member 616b. The electrical connection 620c can branch from electrical connection 620 in the manifold 627, extend into the compartment 617c, and into the chemical agent 618c therein, thereby forming a chemical release member 616c.

In an embodiment, the one or more chemical release members 616a-616c can be positioned in substantially the same lateral location (e.g., location between the nanofoil layers running parallel to the layers) in the RCM 610. For example, the one or more chemical release members 616a-616c can be positioned substantially throughout the entirety of one or more lateral dimensions of the RCM 610. In an embodiment, the one or more chemical release members 616a-616c can be positioned in substantially different lateral locations in the RCM 610 (e.g., parallel to the layers), but each spaced by a lateral distance therebetween. For example, the chemical release member 616a can be disposed in a discrete distal portion of the RCM 610, the chemical release member 616b and be disposed in a medial portion of the RCM 610, and the chemical release member 616c can be disposed in a proximal portion of the RCM 610. Such a configuration can allow selective modification of the modifiable implant. For example, in an embodiment, a medial portion of the RCM 610 can be configured to restrict the relative movement between the implant and the embedding tissue of a subject more than a distal portion or a proximal portion. After a certain time has passed, such as enough time for healing, rehabilitation, or the need for replacement, it can desirable to remove the implant. Activation of one or more of the release materials 611a-611c can be carried out to further facilitate withdrawal of an implant. Any of the chemical release members 616a-616c can be positioned and configured in a similar or identical way as any of the chemical release members 116, 416 disclosed therein.

In an embodiment, a release member including a resistive member, such as any described herein, can be used in place of one or more of the chemical release members 616a-616c. In an embodiment, the release material 611a-611c can include one or more of both of a resistive member or a chemical release member. One or more resistive members can be disposed and positioned within the RCM 610 in a similar or identical way as the chemical release members 616a-616c.

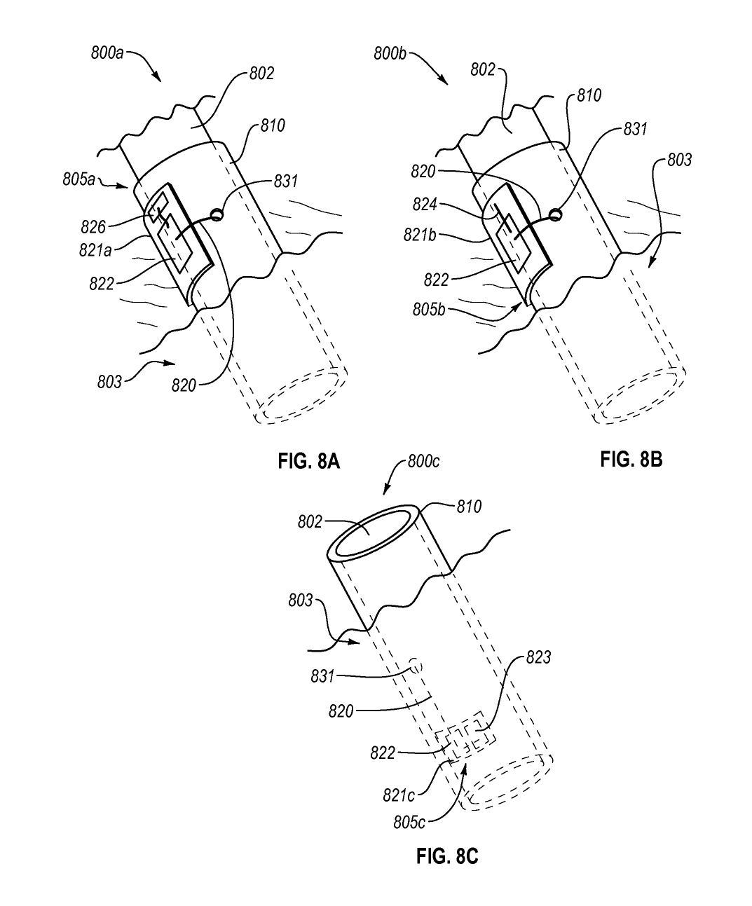

FIGS. 7A-7D depict modifiable implants before and after use according to a number of different embodiment. FIG. 7A is an isometric view of the modifiable implant 700. The modifiable implant 700 can include a body 702 and a release member 705. The release member 705 can be disposed on or in a portion of the body 702, such as extending around at least a portion of an outer (e.g., lateral) surface thereof. The body 702 and the release member 705 can be configured identically or similar to any body or release member herein, including one or more components thereof. In an embodiment, the release member 705 can include one or more surface features 715 thereon. The surface features 715 can be configured to provide the implant with a desired structural association with the embedding tissue, such as protrusions or indentations in the outer surface of the release member 705 to provide an anchor in the embedding tissue. The protrusions or indentations of the one or more surface features can extend a distance outward or inward from the outer surface of the RCM 710. The surface features 715 can protrude or indent about 5% or more of a total width of the RCM outward or inward, such as about 5% to about 100%, about 10% to about 50%, or about 20% of the total width of the RCM 710 outward or inward. The protrusions or indentations of the surface features 715 can be configured as posts, indentations (e.g., divots), ridges (e.g., threads as shown in FIG. 7A), bulges, valleys, or any other suitable surface relief. Such a configuration can aid in anchoring the modifiable implant into embedding tissue of a subject.

The release member 705 can include a circuit 721, RCM 710, and a release material. The circuit 721, RCM 710, and release material 711 can be configured identically or similar to any circuit, RCM, or release material herein, including one or more components thereof.

FIGS. 7B-7D are cross-sectional views of the modifiable implant 700 according to embodiments. FIG. 7B is a cross sectional view of the modifiable implant 700 of FIG. 7A prior to activation of the release member 705. The body 702 is located in the center of the modifiable implant 700. The RCM 710 can be located adjacent to the body, extending substantially completely thereabout (e.g., circumferentially about at least a portion of the outer surface of the body 702). The RCM 710 can include a first layer 716, a second layer 717, and a third layer 718. Any of the layers 716-718 can include any of the materials disclosed herein for RCMs, such as one or more of reactive nanofoil, a resistive member, at least a portion of a chemical release member, a protective layer, or any other suitable material. The release member 705 of the modifiable implant 700 can include a release material 711 therein. The release member 705 can include the circuit 721, an electrical connection 720, and, in an embodiment, the release material 711 therein. The release material 711 can include a resistive member 714 or chemical release member (not shown). The circuit 721 can be similar or identical to any circuit herein.

As shown in FIG. 7B, the first and third layers 716 and 718 of the RCM 710 can be reactive nanofoil layers and the second layer 717 can include a resistive member 714 therein. The resistive member 714 can provide stimulus (e.g., heat or electric ignition) sufficient to initiate reaction of the second layer 717 (e.g., resistive member or chemical agent) and reactive nanofoil of the first and third layers 716 and 718. As shown in FIG. 7C, after activation of the release member 705 and release material 711, the RCM 710 can be at least partially dissociated leaving substantially only the modified implant 700c. The modified implant 700c can include the body 702, circuit 721, and electrical connection 720. In an embodiment, the reaction of the RCM 710 can at least partially dissociate at least a portion of one or more of the circuit 721 or the electrical connection 720. The clearance left by the dissociated RCM 710 can allow the modified implant 700c to be withdrawn from the subject tissue without damaging the implant or surrounding tissue.

As shown in FIGS. 7B and 7D, the first and third layers 716 and 718 of the RCM 710 can be protective layers and the second layer 717 can include reactive nanofoil therein, such that activation of release member 705 dissociates substantially only the second layer 717. The reactive nanofoil in the second layer 717 can be operably coupled to electrical connection 720. The electrical connection 720 can provide stimulus (e.g., heat or electric ignition) sufficient to initiate reaction of the reactive nanofoil in the second layer 717. After activation of the release member 705 and thereby the release material 711, the RCM 710 can be at least partially dissociated leaving substantially only the body 702, circuit 721, or electrical connection 720. In an embodiment, the reaction of the RCM 710 can at least partially dissociate at least a portion of one or more of the circuit 721 or the electrical connection 720. The clearance left by the dissociated second layer 717 can allow the modified implant 700d to be withdrawn from the subject tissue without damaging the implant or surrounding tissue. The first layer 716 can remain substantially secured to the body 702 during withdrawal, and the third layer 718 can remain in the subject, such as until the third layer 718 is removed via crushing or bending the remaining protective layer to facilitate withdrawal. The thickness or number of layers of reactive nanofoil, protective layers, or release materials can be configured to provide a desired gap between the first and third layers 716 and 718 of the modifiable implant 700. Any of the release materials, RCMs, or circuits disclosed herein can be used in conjunction with the modifiable implant 700.

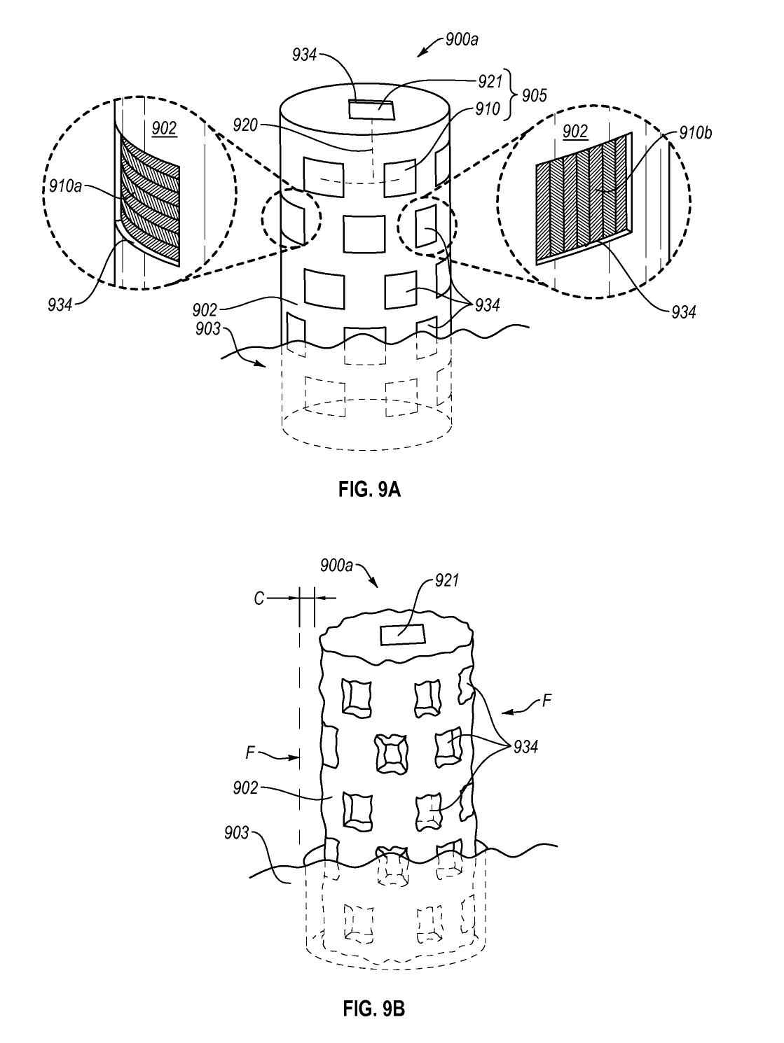

FIGS. 8A-8C are isometric views of embodiments of modifiable implants 800a-800c. The circuit of any of the modifiable implants herein can be associated with the modifiable implant in any number of configurations, including disposed on the surface of the RCM, at least partially disposed within one or more layers of the RCM, on a surface of the body of the implant, at least partially embedded in or internal to the body of the implant, or in surrounding tissue of a subject.

As shown in FIG. 8A, a circuit 821a can be associated with the modifiable implant 800a such as disposed on the surface of the modifiable implant 800a. The modifiable implant 800a can include a body 802, a release member 805a including a circuit 821a, RCM 810, and release material (not shown), each respectively similar or identical to any disclosed herein or portions thereof. The circuit 821a can be operably coupled to one or more layers of the RCM 810 through electrical connection 820 in a conduit 831. The conduit 831 can be a hole in one or more layers of the RCM 810 or a tubing (e.g., insulated housing) extending through one or more layers of the RCM 810 to a portion therein selected for initiating a reaction (e.g., thermal or chemical), such as the release material. The modifiable implant 800a can be implanted in a subject 803. The circuit 821a can be disposed on the surface of the RCM 810. The circuit can include a battery 826 operably coupled to a capacitor 822. In an embodiment, the circuit 821a can include one or more electrical switches therein. Upon activation of the release material, the circuit 821a can be withdrawn from the tissue, such as at the time the body 802 is withdrawn from the tissue of the subject 803.

As shown in FIG. 8B, the circuit 821b can be associated with the modifiable implant 800b such as disposed on the surface of the modifiable implant 800b. The modifiable implant 800b can include the body 802, a release member 805b including a circuit 821b, the RCM 810 and release material, each respectively similar or identical to any disclosed herein or portions thereof. The release member 805b can include a circuit 821b operably coupled to one or more layers of the RCM 810 through electrical connection 820 in the conduit 831. The modifiable implant 800b can be implanted in a subject 803. The circuit 821b can be disposed on the surface of the RCM 810. The circuit 821b can include an antenna 824 or resonator (not shown) operably coupled to a capacitor 822. The circuit 821b, antenna 824, or resonator can be similar or identical to any circuit, antenna, or resonator described herein. In an embodiment, the circuit 821b can include one or more switches therein. Upon activation of the release material, the circuit 821a can be withdrawn from the tissue, such as at the time the body 802 is withdrawn from the tissue of the subject 803.

As shown in FIG. 8C, a circuit 821c can be associated with the modifiable implant 800c such as disposed internal to the surface of the modifiable implant 800c. The modifiable implant 800c can include the body 802 and a release member 805c. The release member 805c can include a circuit 821c, a RCM 810, and release material (not shown), each respectively similar or identical to any disclosed herein or portions thereof. The release member 805c can include a circuit 821c operably coupled to one or more layers of the RCM 810 through electrical connection 820 in the conduit 831. The modifiable implant 800c can be implanted in a subject 803. The circuit 821c can be disposed internal to the surface of the RCM 810. For example, the circuit 821c can be at least partially disposed internally (e.g., embedded on the surface of or disposed entirely within) to the body 802, on the surface of the body 802 and covered by the RCM 810, or internal to the RCM 810 (e.g., between layers therein). The circuit can include a battery 826 operably coupled to capacitor 822. In an embodiment, the circuit 821c can include one or more switches therein. Upon activation of the release material, the circuit 821c can be withdrawn from the tissue with the body 802.

A circuit can be implanted within the subject 803 but not within the embedding tissue as shown in FIGS. 8A and 8B, or the circuit can be implanted within the embedding tissue of the subject 803 as shown in FIG. 8C.

In an embodiment, the release member or a component thereof (e.g., release material or RCM) can extend about the entire body or only a portion of the body, such as about (e.g., circumferentially) only a portion of the outer surface of the body. For example, the RCM can extend about only a portion of a dimension or surface of the body, such as more than about 5%, about 5% to about 95%, about 10% to about 80%, about 25% to about 75%, about 40% to about 60%, about 20% to about 40%, about 50% to about 90%, about 25%, or about 50% of the outer surface of the body. The release material (e.g., resistive member or chemical release member) can include any desired lateral dimensions ranging from 1% of a lateral dimension of the RCM to 100% of a lateral of the RCM, such as about 1% or more, about 2% to about 90%, about 5% to about 80%, about 10% to about 75%, about 25%, to about 50%, about 5%, about 10%, about 20%, or less than about 90% of a lateral of the RCM. The release material can be disposed in any portion of the RCM. The release material can be positioned in the RCM at an intermediate point therein. The release material can be located equidistantly from the ends of the RCM or closer to one end.

While the bodies in FIGS. 8A-8C are depicted as linear shafts, bodies can exhibit any geometric configurations encountered in implants (e.g., devices or artificial biological structures). For example, the body can be a pin, a post, bracket, an artificial bone, a pump, a pacemaker, or a screw having a shaft and head. In an embodiment, the body can include more than one member, such as in a joint or plurality of bones. In an embodiment, the first body member can be a ball of a joint and the second body member can a socket of a joint. In an embodiment, the first body member can be at least a portion of an artificial vertebral bone and the second body member can be at least a portion of an adjacent artificial vertebral bone. The associated release members can be configured to match the geometry of the at least one body member.

In an embodiment, more than one RCM can be used, such as more than one RCM about a single implant, or a plurality of RCMs can be used on a plurality of implants, such as one or more RCMs about each implant of a plurality of implants.

In an embodiment, an implant can include a plurality of RCM portions, (e.g., stacks) at least some of which are integral to (e.g., embedded in the surface or internal to) the body and spaced from each other a distance. Each of the RCMs can include a portion of a single collective release member or can each include one or more of a plurality of release members, similar or identical to any release member disclosed herein. Each of the plurality of release members can be configured to be selectively activated, such as having a unique frequency, wavelength, electromagnetic radiation polarity, or encryption associated therewith. In such embodiments, activating the release member or release material therein to at least partially dissociate the RCM can include selectively activating at least some of the plurality of release members. Selectively activating at least some of the plurality of release members can alter a compliance or flexibility of the implant collectively or any of the individual members therein. Upon activation of at least some of the plurality of release members, the structural connectivity of the implant can be altered by compressive, bending, shear, or tensile forces placed on the body. For example, the structural rigidity of a body can be reduced when the volume of space therein is empty after activation of the release member (e.g., release material) and partially dissociation of the RCM therein. Such an embodiment can provide a selectively modifiable structural flexibility and can be susceptible to crushing, bending, or other forces. In an embodiment, such increased compliance or flexibility can be desirable. Accordingly, only some of or all of the plurality of release members/material can be activated at differing times, or the same time.

In an embodiment, the RCM associated with body can be disposed in an interior portion of the body and can be configured to allow at least one of the members to release from an embedding tissue upon activation of the release member. For example, the body can be released from the embedding tissue by activating one or more release materials in RCMs internal thereto, thereby collapsing or allowing crushing of the body. Such collapsing or crushing can be effective to provide sufficient clearance for the body to be withdrawn from an embedding tissue.

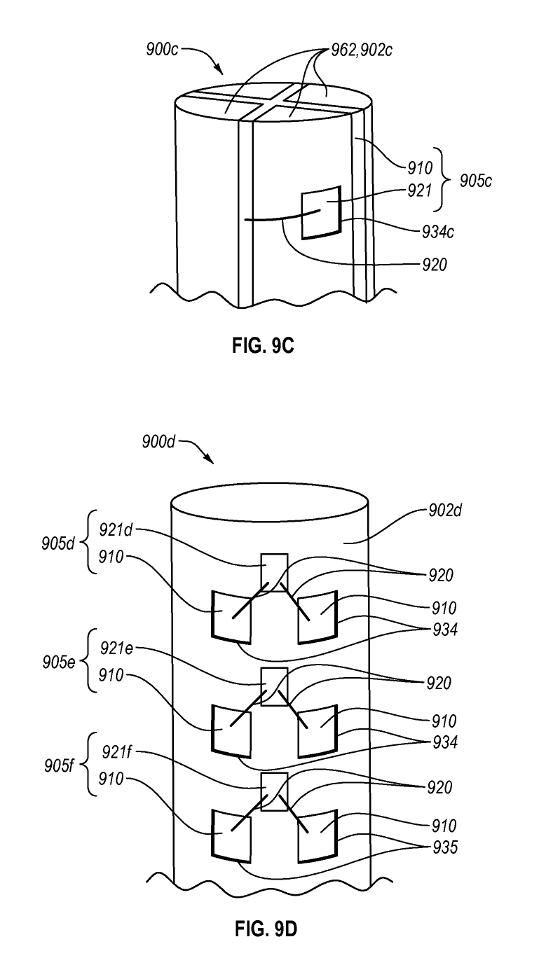

FIGS. 9A-9C are isometric views of modifiable implants having at least a portion of one or more release members (e.g., RCMs) therein. In an embodiment, the body can be configured to allow crushing, compressive, tensile, or other forces to deform the body if no additional support is provided to the body.

For example, as shown in FIG. 9A, the implant 900a includes a body 902 that includes a plurality of cavities 934 therein. Each of the plurality of cavities 934 can include a portion of one or more release members 905 therein, such as one or more of an RCM 910 (e.g., RCM stack) or circuit 921 therein. The cavities 934 can extend from the surface of the body 902 inward a distance. The one or more cavities 934 can be entirely within the surface of (e.g., entirely internal to) the body 902. In an embodiment, one or more of the cavities 934 can extend entirely through the body 902 or less, such as about 5% of a dimension (e.g., lateral or longitudinal) of the body or more, such as about 5%, about 100%, about 10% to about 80%, about 25% to about 75%, about 40% to about 60%, about 50% to about 95%, about 20%, about 40%, about 60%, or about 50% or less of a dimension of the body 902. Suitable geometric configurations for the cavities 934 can include one or more concentrically shaped portions (concentrically shaped to the body) therein, polygonal shaped portions (e.g., cube, rectangle), cylinders, slices or wedges, longitudinal cross-sections thereof, a latitudinal cross-sections thereof, amorphous shapes, or combinations of any of the foregoing. In an embodiment, the cavities 934 can exhibit substantially uniform shapes, such as rectangular prismatic, spherical, tubular, conical, polygonal, or combinations thereof. In an embodiment, one or more of the cavities 934 can exhibit substantially non-uniform shapes such as amorphous shapes; shapes having varying, non-repeating cross-sectional thicknesses or dimensions; or shapes having no cross-sectional symmetry.

The one or more cavities 934 can represent at least about 5% or more of the volume of the implant 900a, such as about 5% to about 90%, about 10% to about 80%, about 25% to about 75%, about 40% to about 60%, about 50%, about 80% or less, or about 33% of the volume of the implant 900a.

One or more of the cavities 934 can include the RCM 910 therein, such as any RCM disclosed herein. For example, RCMs 910 can include RCM stacks. In an embodiment, the RCMs 910 can be stacked or layered in the cavities 934 in any number of directions. For example, the RCM 910a can include a longitudinally stacked configuration (e.g., axially stacked horizontal layers) and the RCM 910b can include a laterally stacked configuration (e.g., vertical layers). In an embodiment, the RCM 910 can be stacked radially with respect to the body 902. In an embodiment, the RCM 910 can be rolled up into a substantially cylindrical shape and placed into a cavity 934. The RCM 910 can exhibit a substantially uniform shape to the cavity 934 thereby substantially filling the entire cavity 934. In an embodiment, the RCM 910 need not fill the entire cavity 934, such as a cylindrical RCM 910 in a rectangular cavity.

One or more of the cavities 934 can include a circuit 921 therein. The circuit 921 can be operably coupled to one or more of the RCMs 910 in the cavities 934, such as through electrical connections 920 and conduits thereto.

In embodiments where the cavities 934 are entirely internal to the body 902, the body 902 can also include ports or vents extending from each of the cavities to the surface of the body 902 or a chamber internal to the body, such that any reactants or pressure developed during the reaction of the RCM can be vented without causing the implant to expand or explode. In an embodiment (not shown), substantially the entire interior of the body 902 can include a cavity 934 and an RCM therein (e.g., RCM filling a hollow cylinder), such that upon dissolution of the RCM, the body 902 may be crushed, compacted, bent, or otherwise manipulated to facilitate removal. In such embodiments, the RCM can provide the selected structural rigidity to the implant until removal is required.

As shown in FIG. 9B, after the release material has been activated and the RCMs 910 in the cavities have be substantially dissociated, the now empty cavities 934 can allow the body 902 to collapse inward or be crushed by a force F, such as from surrounding tissue or a medical technician. The implant 900a, now having the collapsed or crushed body 902b, can exhibit a clearance C between the body 902c and subject 903 effective to allow the body 902c to be withdrawn from the subject 903 without damage or trauma to the embedding tissue of the subject 903.

FIG. 9C is an isometric view of a modifiable implant 900c according to an embodiment. In an embodiment, a cavity 934c can include a space between separate portions of a body, such as individual, non-interfacing sections 962 of a body 902c. For example, the body 902c can include one or more wedge shaped sections 962 at least partially separated by RCMs 910 therebetween. The sections 962 can extend in a longitudinal direction and the RCM 910 can extend therebetween. The release member 905c can include at least one RCM 910 and one or more circuits 921. The circuit 921 can be configured similar or identical to any circuit herein. The circuit 921 can be positioned on the surface of the body, in the RCM 910, or embedded in a separate cavity in one or more sections 962 of the body 902c. The RCM 910 can be similar or identical to any RCM herein. The RCM 910 can include an adhesive effective to bond the sections 962 together, thereby forming a single body 902c. As shown, at least one RCM 910 can extend between each section 962 and provide structure to the overall modifiable implant 900c. The at least one RCM 910 can be 5% or more of a total volume of the modifiable implant 900c, such as about 5% or more of the volume, such as about 5% to about 90%, about 10% to about 50%, about 25% to about 75%, about 20% to about 40%, about 10% to about 30%, about 80% or less of the volume of the modifiable implant 900c. While shown as a cross between the sections 962, the at least one RCM 910 can be configured in any suitable configuration, such as slices (e.g., in a stacked section-RCM-section configuration), columns, cylinders, or any other conformation configured to provide structural connectivity to one or more sections 962.

FIG. 9D is an isometric view of a modifiable implant 900d. In an embodiment, a modifiable implant can include more than one release member. Each release member can be configured to be activated independently. The modifiable implant 900d can include a body 902d and a plurality of release members 905d-905f. Each of the release members 905d-905f can include RCM 910 and a corresponding circuit 921d-921f operably connected thereto by one or more electrical connections 920. The RCMs 910 can be positioned in or on a portion of the body 902d. For example, the body 902d can have one or more cavities 934 therein, with each cavity having an RCM 910 therein. Each of the circuits 921d-921f can be configured similar or identical to any circuit herein. Each of the circuits 921d-921f can be configured to activate responsive to the same stimulus to a separate stimulus. For example, the circuit 921d can be configured to activate upon receiving a first stimulus, the circuit 921e can be configured to activate upon receiving a second stimulus, and the circuit 921f can be configured to activate upon receiving a third stimulus. In an embodiment, the first, second, and third stimuli can include one or more of differing electromagnetic radiation frequencies, differing electromagnetic radiation wavelengths, differing electromagnetic radiation polarities, differing radio frequencies, differing radio frequency wavelengths, differing radio frequency amplitudes, differing infrared wavelengths, differing infrared frequencies, differing sonic wavelengths, differing sonic frequencies, or differing magnetic fields, or one of each of the foregoing. The release members 905d-905f can be selectively activated by providing the stimulus corresponding to the particular release member.

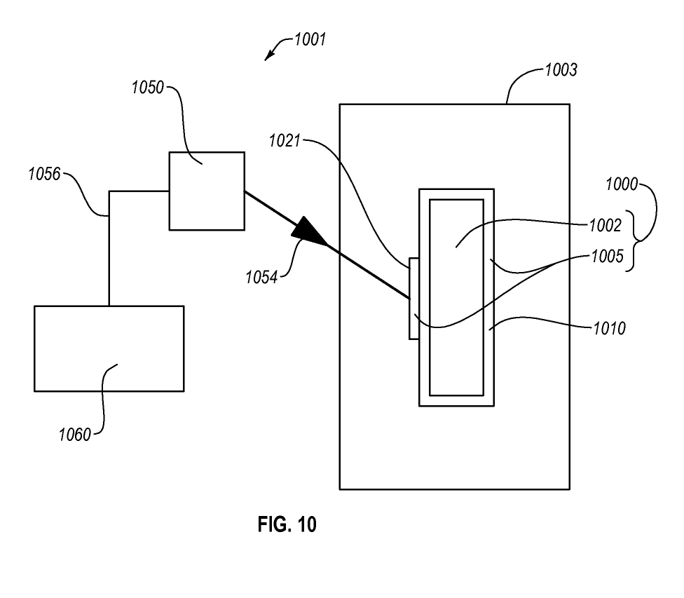

FIG. 10 is schematic diagram of a system 1001 for modifying an implant according to an embodiment. The system 1001 can include one or more stimulus sources 1050 and one or more modifiable implants 1000. The modifiable implant 1000 can be implanted within or on the subject 1003. The modifiable implant 1000 can be configured similarly or identical to any modifiable implant described herein, including any components thereof. In an embodiment, the modifiable implant 1000 can include a body 1002 and a release member 1005. The release member 1005 can be similar or identical to any release member disclosed herein, including any associated release material, circuit, or RCM disclosed herein. In an embodiment, release member 1005 of the modifiable implant 1000 can include a circuit 1021 and RCM 1010. The RCM 1010 can be configured similar or identical to any RCM disclosed herein, including any components thereof or any configurations thereof (e.g., layers, materials, stacks, etc.).

In an embodiment, the release member 1005 or one or more components thereof therein can extend entirely around a lateral surface of the body 1002. The release member 1005 can include a release material similar or identical to any release material disclosed herein. In an embodiment, the release member 1005 can include a chemical release member operably coupled to the circuit 1021 by an electrical connection 1020. The circuit 1021 can be configured similar to any circuit disclosed herein. For example, the circuit 1021 can include one or more of a capacitor, electrical connection, battery, antenna, one or more resonators, switch, resistive member, or chemical release member similar or identical to any disclosed herein. In an embodiment, the chemical release member can include a chemical agent (not shown) disposed between one or more layers of the RCM 1010. The chemical agent can be in communication with the electrical connection (not shown), sufficient to allow an electrical current therethrough to trigger a reaction including the chemical agent. In an embodiment, the chemical release member can include a chemical agent releasably stored in a compartment therein. The chemical agent can include a composition configured to degrade or otherwise dissociate the RCM 1010. The chemical release member can be operably coupled to the circuit 1021. The circuit 1021 can be configured to emit an electrical charge effective to cause the compartment to rupture and release the chemical agent therein.

In an embodiment, the release member 1005 can include a resistive member operably coupled to a circuit having a capacitor and an antenna (e.g., an electrical release member) having or coupled to a resonator (not shown). The capacitor can be configured to be charged via radio frequency energy directed to the antenna or resonator and discharged through a resistive member (not shown) operably coupled to the capacitor. The resistive member can heat-up causing one or more layers (e.g., reactive nanofoil) of the RCM 1010 to react and at least partially dissociate or degrade. In an embodiment, the release member 1005 can include a resistive member operably coupled to a circuit having a capacitor and battery (e.g., an electrical release member). The capacitor can be configured to be charged via battery and discharged through a resistive member (not shown) operably coupled to the capacitor.

The stimulus source 1050 can be configured to provide a stimulus 1054 to the release member 1005 effective to cause activation of the release member or release material therein. For example, the stimulus source 1050 can be configured as an electromagnetic radiation generator, such as a radio frequency signal generator or a microwave generator; an electromagnetic field generator; or a sonic vibration (e.g., ultrasound or acoustic) generator. In an embodiment, the stimulus source 1050 can be a radio frequency generator configured to send one or more specific frequencies (e.g., narrow-band frequency), amplitudes, or wavelengths of radio frequency radiation to one or more release members or release materials. The radio frequency generator can be configured to selectively send a specific frequency or wavelength depending on the desired modification to the structural connection of the modifiable implant. For example, a first radio frequency can trigger a first release material or a portion thereof, and a second radio frequency can trigger a second release material or a portion thereof. The effective range of the stimulus source 1050 can depend on the type of stimulus 1054, the size of the components of the release member 1005, or the type and location of the modifiable implant 1000 within the subject 1003. Effective ranges can include at least those ranges inside the same room, doctor's office, or medical facility.

In an embodiment, the stimulus source 1050 can be operably connected to a controller 1060, such as a computer or tablet. The controller 1060 can be configured to activate, direct, adjust, or provide instructions to the stimulus source 1050. For example, the controller 1060 can be a computer configured to control at least one characteristic (e.g., frequency, wavelength, duration, etc.) of the stimulus 1054 generated by the stimulus source 1050. The stimulus source 1050 can be operably coupled to the controller 1060 via an operable connection 1056. The operable connection 1056 can include one or more of a wireless connection or a physical electrical connection such as wiring.

In an embodiment, the release member 1005 can have different configurations, some of which can include one or more batteries, antennas, timers, electrical switches, or capacitors as described herein. In an embodiment, the circuit 1021 can include one or more timers (not shown) therein. The one or more timers can be configured to close a circuit between a battery and the capacitor sufficient to allow the capacitor to charge after a selected amount of time has elapsed. The one or more timers can be configured to close a circuit between the capacitor and the release material to discharge therein after a selected amount of time has elapsed. The one or more timers can be preprogrammed to cause an electrical switch to close after a selected time, such as based on a forecasted healing time or rehabilitation schedule (e.g., 1 day or more, 1 week, 1 month, or 1 month or more). In an embodiment, one or more timers can be included in any release member disclosed herein.

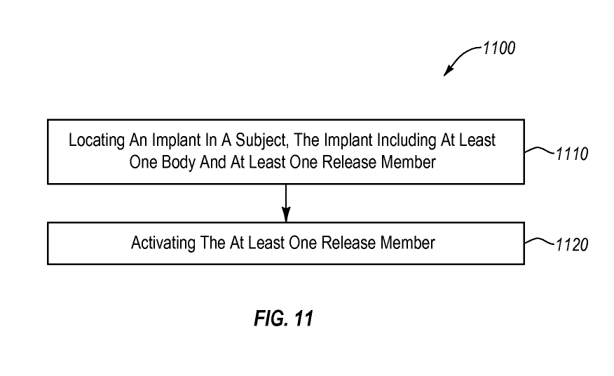

FIG. 11 is a schematic flow diagram of a method 1100 of modifying a modifiable implant according to an embodiment. The method 1100 can include the act 1110 of locating a modifiable implant in a subject. Locating the implant can include locating a general area that the implant is located in, such as by visual detection (e.g. locating scar tissue from implantation surgery), palpation, reviewing a chart, or via medical scans (e.g., X-ray, CT scans, etc.). The implant can be similar or identical to any modifiable implant disclosed herein. For example, the implant can include a body and at least one release member associated therewith. The release member can include at least one section of RCM and at least one release material associated therewith. The at least one release material can be configured to at least partially alter the structural connectivity (e.g., dissociate) of the at least one release member to facilitate (e.g., ease) removal of the implant from the subject. For example, the at least one release material can be configured to provide a clearance between the body of the implant and the tissue of a subject upon activation thereof. In an embodiment, the change in structural connectivity of the at least one release member (e.g., from within one or more cavities) can allow the body of the implant to be crushed, bent, or otherwise manipulated to facilitate withdrawal from embedding tissue.

The method 1100 can additionally or alternatively include an act of positioning the implant in or on a subject. The implant can be similar or identical to any modifiable implant disclosed herein. Positioning the implant in or on a subject can include surgical implantation, adhesion, or any other suitable means of placing an implant in or on a subject.

The method 1100 can include the act 1120 of activating the release member. The act 1120 can include activating at least one release member (e.g., to at least partially dissociate the RCM between the body and the subject) effective to allow the implant to break free of the subject or easily be removed from the subject. Activating the at least one release member can include activating the at least one release material therein. The implant can break free of the subject by providing a gap or space between the subject and the body of the implant upon activation of the at least one release member. In an embodiment, activating the at least one release member to at least partially dissociate the RCM can include causing a change in the physical state of at least a portion of the RCM, such at least one of a solid-to-liquid transition, a solid-to-gas transition, a gel-to-liquid transition, a gel-to-gas transition, a foam-to-liquid transition, or a foam-to-gas transition.

In an embodiment, activating the release member to at least partially dissociate the RCM can include directing a stimulus such as electromagnetic radiation (e.g., radio frequency radiation or infrared radiation), magnetic force, or sonic vibrations at the release member such as the circuit or antenna of the release member. In an embodiment, activating the release member to at least partially dissociate the RCM can include releasing a chemical agent and exposing the RCM (e.g., reactive nanofoil) to the chemical agent. In an embodiment, activating the release member includes providing an electrical charge to the chemical agent via a capacitor, a battery, or a radio frequency antenna operably coupled thereto.

In an embodiment, providing a stimulus to the release member can include directing the stimulus to one or more electrical switches in the release member, the one or more electrical switches can be configured to open or close a circuit or connection to a battery or capacitor as described herein. The one or more electrical switches can include an antenna, the antenna can collect and convert the stimulus into electrical charge and deliver the electrical charge to the electrical switches operably coupled thereto to open or close the one or more switches.

In an embodiment, the stimulus can include radio frequency radiation and providing a stimulus to the at least one release member can include directing the radio frequency radiation to an antenna of the circuit of the release member. Directing the radio frequency radiation to an antenna can include collecting and converting the radio frequency radiation into electrical charge at the antenna and delivering the electrical charge to a capacitor operably coupled thereto. The capacitor can deliver the electrical charge to the resistive member effective to dissociate (e.g., dissolve, melt, or chemically react) the at least one release member. One or more antennas can be configured to receive the radio frequency radiation over a selected frequency range. In an embodiment, the method 1100 can including removing the body from the subject subsequent to activating the at least one release member.

In an embodiment, the at least one release member can include a chemical release member having a chemical agent therein. The chemical agent can be composed to dissociate or degrade the reactive composite material when exposed thereto. The release member can include a circuit configured to emit an electrical charge effective to initiate reaction of the chemical agent in the chemical release member. The circuit can be configured according to any circuit disclosed herein. In an embodiment, activating the at least one release member can include providing the electrical charge effective to initiate reaction of the release material therein (e.g., chemical agent or resistive member), such as via an electrical charge from the capacitor or a radio frequency antenna or battery operably coupled thereto.

In an embodiment, the body can include one or more cavities therein; the one or more cavities can positioned and configured to promote release of the implant as disclosed herein. At least a portion of one or more release members can be disposed in one or more of the cavities. The one or more release members can include RCMs or stacks thereof as described herein. In such embodiments, activating the at least one release member can include selectively activating at least one release member of a plurality of release members. Activation of the release members can include activation of the release materials associated therewith.

In an embodiment, one or more antennas can be configured to receive electromagnetic radiation such as radio frequency radiation, over a selected frequency range. The act of providing a stimulus to the release member can include directing the electromagnetic radiation to the release member at a frequency within the frequency range that the antenna is configured to receive. In an embodiment, the act of providing the stimulus can include providing an encrypted or otherwise encoded stimulus, such as a frequency inverted radio signal or narrow-band radio frequency to an antenna or resonator (e.g., narrow-band resonator) in a release member.

Each of a plurality of release members can be configured to be selectively activated, such as having a unique frequency, wavelength, or encryption associated therewith. In such embodiments, activating the release member to at least partially dissociate the RCM can include selectively activating at least some of the plurality of release members. Selectively activating at least some of the plurality of release members can alter a compliance or flexibility of the implant collectively or any of the individual members therein. Upon activation of at least some of the plurality of release members, the structural connectivity of the implant can be altered by compressive, bending, shear, or tensile forces placed on the body. For example, the structural rigidity of a member can be reduced when the volume of space therein is empty after activation of the release member and partially dissociation of the RCM therein. Such an embodiment can have a selectively modifiable structural flexibility and can be susceptible to crushing, bending, or other forces. In an embodiment, such increased compliance or flexibility can be desirable. Accordingly, only some or all of the plurality of release members can be activated at differing times, or the same time.

In an embodiment, activation of the release member can be configured to selectively permit lateral motion along an interface between the body and the subject. In an embodiment, activation of the release member can be configured to selectively permit axial motion along an interface between the body and the subject.

While the examples of the modifiable implants herein are provided in a biological context, non-biological uses are also considered. For example, in a mechanical structure, it may be desirable to temporarily lock an implant in place to allow curing time for portions of the structure prior to full use of the implant. In other embodiments, installation of a mechanical part can require the part to have a specific conformation during installation but require free movement during use. In an embodiment, the modifiable implant can be a mechanical fastener split into more than one member (e.g., surgical or industrial screw or bolt). For example, the first member can be configured as a threaded shank portion and the second member can be configured as a head portion with an RCM at least partially bonding the first and second members together. The head portion can be dissociable from the threaded portion via actuation of the release member in the RCM. A suitable “implantable” mechanical structure can include an RCM and members substantially similar to any disclosed herein whether used in a biological subject or otherwise.

The reader will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. The reader will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. The reader will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, the various embodiments described herein can be implemented, individually and/or collectively, by various types of electro-mechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein may be implemented in an analog or digital fashion or some combination thereof.

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In an embodiment, the modifiable implants and systems for modifying an implant disclosed herein can be integrated in such a manner that the modifiable implants and systems operate as a unique system configured specifically for the function of structurally or otherwise modifying the implant, and any associated computing devices of the modifiable implants and systems operate as specific use computers for purposes of the claimed system, and not general use computers. In an embodiment, at least one associated computing device of the modifiable implants and systems operate as specific use computers for purposes of the claimed system, and not general use computers. In an embodiment, at least one of the associated computing devices of the modifiable implants and systems are hardwired with a specific ROM to instruct the at least one computing device. In an embodiment, one of skill in the art recognizes that the modifiable implants and systems effects an improvement at least in the technological field of implants.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components may be referred to herein as “configured to.” The reader will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, the recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

What is claimed is:

1. A modifiable implant, comprising:
at least one body configured to be implanted in a subject; and
at least one release member disposed on at least a portion of the at least one body, the at least one release member including,
a reactive composite material; and
at least one release material associated with the reactive composite material, the at least one release material configured to at least partially alter at least a structural connectivity of the at least one release member.

2. The modifiable implant of claim 1, wherein the at least one release member is configured to change a physical state of the reactive composite material.

3. The modifiable implant of claim 2, wherein the physical state changes from a solid to a liquid.

4. The modifiable implant of claim 2, wherein the physical state changes from a solid to a gas.

5. The modifiable implant of claim 2, wherein the reactive composite material includes a phase change material, and wherein the at least one release member is configured to change the physical state of the phase change material.

6. The modifiable implant of claim 1, wherein the at least one release member is configured to initiate a chemical reaction between two or more components of the reactive composite material upon activation.

7. The modifiable implant of claim 1, wherein the reactive composite material includes at least one layer of reactive nanofoil.

8. The modifiable implant of claim 1, wherein the at least one release member extends about at least a portion of an exterior surface of the at least one body and is configured to at least partially dissociate the reactive composite material to facilitate withdrawal of the at least one body from the tissue of the subject.

9. The modifiable implant of claim 1, wherein the reactive composite material includes a plurality of layers including one or more of at least one reactive nanofoil, at least a portion of the at least one release material, or at least one protective layer.

10. The modifiable implant of claim 9, wherein the reactive composite material includes a plurality of reactive nanofoil layers having at least a portion of the at least one release material disposed therebetween.

11. The modifiable implant of claim 1, wherein the at least one release material includes one or more of a resistive member or a chemical release member.

12. The modifiable implant of claim 11, wherein the at least one release member includes a resistive member operatively coupled to a circuit having a capacitor and an antenna, and wherein the capacitor is configured to be charged via radio frequency radiation, ultrasonic vibrations, or infrared light directed to the antenna and discharged through the resistive member.

13. The modifiable implant of claim 11, wherein the at least one release member includes a resistive member operatively coupled to a circuit having a capacitor and a battery, and wherein the capacitor is configured to be charged via the battery and discharged through the resistive member.

14. The modifiable implant of claim 11, wherein:
the at least one release material includes a chemical release member having a chemical agent layer, the chemical agent layer composed to degrade the reactive composite material when exposed thereto; and
the at least one release member includes a circuit configured to emit an electrical charge effective to initiate reaction of the chemical agent.

15. The modifiable implant of claim 11, wherein:
the at least one release material includes a chemical release member having a chemical agent releasably stored in a compartment therein, the chemical agent being composed to degrade the reactive composite material when exposed thereto; and
the at least one release member includes a circuit configured to emit an electrical charge effective to rupture the compartment and release the chemical agent therein.

16. The modifiable implant of claim 1, wherein the at least one release member extends about at least a portion of an exterior surface of the at least one body, the at least one release member including the at least one release material disposed between the exterior surface of the at least one body and the reactive composite material.

17. The modifiable implant of claim 1, wherein the at least one body is configured as a screw, post, pin, bracket, drug delivery device, pacemaker, or plate.

18. The modifiable implant of claim 1, wherein:
the at least one body includes one or more cavities therein, the one or more cavities positioned and configured to promote release of the at least one body by allowing the at least one body to collapse inward responsive to one or more forces thereon; and
at least a portion of one or more of the at least one release member is disposed in the one or more cavities.

19. (canceled)

20. The modifiable implant of claim 18, wherein the reactive composite material includes a plurality of reactive nanofoil layers having at least a portion of one or more release materials therebetween.

21. The modifiable implant of claim 18, wherein the at least one release member is positioned in one of the one or more cavities and includes a plurality of layers stacked radially with respect to the at least one body.

22. The modifiable implant of claim 18, wherein the at least one release member is positioned in one of the one or more cavities and includes a plurality of layers stacked longitudinally with respect to the at least one body.

23. The modifiable implant of claim 18, wherein the at least one release member is positioned in one of the one or more cavities and includes a plurality of layers stacked laterally with respect to the at least one body.

24. The modifiable implant of claim 18, wherein the reactive composite material includes a plurality of layers including one or more of at least one reactive nanofoil, at least a portion of the at least one release material, or a protective layer.

25. The modifiable implant of claim 18, wherein the at least one release material includes one or more of resistive member or a chemical release member.

26. The modifiable implant of claim 18, wherein the at least one release member includes a resistive member operatively coupled to a circuit having a capacitor and an antenna, and wherein capacitor is configured to be charged via radio frequency radiation directed to the antenna and discharged through the resistive member.

27. The modifiable implant of claim 18, wherein the at least one release member includes a resistive member operably coupled to a circuit having a capacitor and a battery, and wherein the capacitor is configured to be charged via the battery and discharged through the resistive member.

28. The modifiable implant of claim 18, wherein the at least one release member includes a circuit operably coupled to a chemical release member having chemical agent therein, the chemical agent composed to degrade the reactive composite material when exposed thereto, the circuit configured to emit an electrical charge effective to initiate reaction of the chemical agent.

29. The modifiable implant of claim 18, wherein the at least one release member includes a circuit operably coupled to a chemical release member having a chemical agent releasably stored in a compartment in the reactive composite material, the chemical agent being composed to degrade the reactive composite material when exposed thereto, the circuit configured to emit an electrical charge effective to rupture the compartment and release the chemical agent therein.

30. The modifiable implant of claim 18, wherein the at least one body is configured as a screw, post, pin, bracket, drug delivery device, pacemaker, or plate.

31. The modifiable implant of claim 1, wherein:
the at least one release member includes a plurality of release members, each of which is internal to the at least one body and spaced from one another;
the at least one release material includes a plurality of release materials each of which is associated with a corresponding one of the plurality of release members; and
each of the plurality of release members is configured to be selectively activated.

32. A method of removing an implant, the method comprising:
locating an implant in a subject, the implant including,
at least one body; and
at least one release member disposed on at least a portion of the at least one body, the at least one release member including,
a reactive composite material;
at least one release material associated with the reactive composite material, the at least one release material configured to at least partially alter the at least one release member; and
activating the at least one release member to facilitate removal of the at least one body from the subject.

33. The method of claim 32, wherein the at least one release material includes one or more of a resistive member or a chemical release member.

34. The method of claim 32, further including removing the at least one body from the subject subsequent to activating the at least one release member.

35. The method of claim 32, wherein:
the reactive composite material includes a phase change material;
the at least one release member is configured to change the physical state of the phase change material; and
activating the release member to facilitate removal of the at least one body from the subject includes causing a change in the physical state of the phase change material.

36. The method of claim 32, wherein:
the at least one release member includes a resistive member operatively coupled to a circuit having a capacitor and an antenna; and
the capacitor is configured to be charged via radio frequency radiation directed to the antenna and discharged through the resistive member operably coupled to the capacitor.

37. The method of claim 32, wherein:
the at least one release member includes a resistive member operably coupled to a circuit having a capacitor and a battery; and
the capacitor is configured to be charged via the battery and discharged through the resistive member operably coupled to the circuit.

38. The method of claim 32, wherein activating the at least one release member includes providing a stimulus to the at least one release member, the stimulus including one or more of radio frequency radiation, ultrasonic vibration, infrared radiation, or a magnetic force.

39. The method of claim 38, wherein the stimulus is encrypted or otherwise encoded.

40. The method of claim 38, wherein:
the stimulus includes radio frequency radiation;
the at least one release member includes a circuit having a capacitor and an antenna operatively coupled thereto, the antenna being configured to receive radio frequency radiation and convert the radio frequency radiation into electrical charge; and
providing a stimulus to the at least one release member includes directing the radio frequency radiation to the antenna of the at least one release member.

41. The method of claim 40, wherein:
the at least one release material extends about at least a portion of an exterior surface of the at least one body; and
activating the at least one release member includes directing radio frequency radiation at the antenna of the circuit effective to cause the at least one release material to at least partially dissociate the reactive composite material.

42. The method of claim 40, wherein the antenna is configured to receive the radio frequency radiation over a selected frequency range.

43. The method of claim 32, wherein:
the at least one release material extends about at least a portion of an exterior surface of the at least one body;
the at least one release member includes a circuit operably couple to a chemical release member having a chemical agent, the chemical agent composed to degrade the reactive composite material when exposed thereto, the circuit configured to emit an electrical charge effective to initiate reaction of the chemical agent; and
activating the at least one release member includes providing the electrical charge effective to initiate reaction of the chemical agent.

44. The method of claim 43, wherein the circuit includes a capacitor or a radio frequency antenna operably coupled thereto; and
providing the electrical charge effective to initiate reaction of the chemical agent includes providing an electrical charge to the at least one release material via a capacitor or a radio frequency antenna operably coupled thereto.

45. The method of claim 32, wherein:
the at least one release member extends about at least a portion of an exterior surface of the at least one body; and
the at least one release member includes a circuit operably coupled to a chemical release member having a chemical agent releasably stored in a compartment, the chemical agent composed to degrade the reactive composite material when exposed thereto, the circuit configured to emit an electrical charge effective to rupture the compartment and release the chemical agent therein; and
activating the at least one release member includes the releasing the chemical agent and exposing the reactive composite material to the chemical agent.

46. The method of claim 45, wherein releasing the chemical agent and exposing the reactive composite material to the chemical agent includes providing the electrical charge to the electrical release circuit via a capacitor or a radio frequency antenna operably coupled thereto.

47. The method of claim 32, wherein the at least one body includes one or more cavities therein, the one or more cavities positioned and configured to promote release of the at least one body by allowing the at least one body to collapse inward responsive to one or more forces thereon, and wherein the at least one release member is disposed in the one or more cavities.

48. (canceled)

49. (canceled)

50. (canceled)

51. (canceled)

52. (canceled)

53. (canceled)

54. The method of claim 47, wherein activating the at least one release member includes directing radio frequency radiation at the at least one release member having a resistive member therein, effective to cause the resistive member to at least partially dissociate the reactive composite material.

55. The method of claim 47, wherein:
the at least one release member includes a circuit operably coupled to a chemical release member having a chemical agent layer, the chemical agent layer composed to degrade the reactive composite material when exposed thereto, the circuit having a capacitor configured to emit an electrical charge effective to initiate reaction of the chemical agent; and
activating the at least one release member includes providing an electrical charge effective to initiate reaction of the chemical agent.

56. The method of claim 55, wherein providing the electrical charge effective to initiate reaction of the chemical agent includes providing the electrical charge from the capacitor via a radio frequency antenna operably coupled thereto.

57. The method of claim 47, wherein the at least one release member includes a circuit operably coupled to a chemical release member having a chemical agent releasably stored in a compartment therein, the chemical agent composed to degrade one or more portions of the reactive composite material when exposed thereto, the circuit having a capacitor configured to emit an electrical charge effective to rupture the compartment and release the chemical agent therein; and
activating the at least one release member includes releasing the chemical agent and exposing the reactive composite material to the chemical agent.

58. The method of claim 57, wherein releasing the chemical agent and exposing the reactive composite material to the chemical agent includes providing the electrical charge from the circuit via the capacitor or a radio frequency antenna operably coupled thereto.

59. The method of claim 32, wherein the at least one body is configured as a screw, post, pin, bracket, drug delivery device, pacemaker, or plate.

60. The method of claim 32, wherein:
at least a portion of the at least one release member is internal to the at least one body;
the at least one release member is configured to be selectively activated; and
activating the at least one release member includes selectively activating the at least one release member.

61. The method of claim 32, wherein:
the at least one release member includes a plurality of release members, each of which is internal to the at least one body, spaced from one another, and configured to be selectively activated; and
activating the at least one release member includes selectively activating one or more of the plurality of release members.

62. A system for modifying an implant, the system comprising:
an implant configured to be implanted in a subject, the implant including,
at least one body; and
at least one release member disposed on at least a portion of the at least one body, the at least one release member including,
a reactive composite material; and
at least one release material associated with the reactive composite material, the at least one release material configured to at least partially alter at least a structural connectivity of the at least one release member; and
a stimulus source configured to provide a stimulus to the at least one release member effective to cause activation thereof.

63. The system of claim 62, wherein the stimulus source includes one or more of an electromagnetic radiation generator, an electromagnetic field generator, or a sonic vibration generator.

64. The system of claim 62, wherein the stimulus source includes an electromagnetic radiation generator configured as a radio frequency generator.

65-85. (canceled)
